(19) United States
(12) Patent Application Publication
(10) Pub. No.: US 2025/0135114 A1
(43) Pub. Date: May 1, 2025

(54) CO-LOCATION CRADLE FOR AUTOMATED MEDICAMENT DELIVERY SYSTEMS AND ANALYTE SENSORS AND DEPLOYMENT THEREOF

(71) Applicant: Insulet Corporation, Acton, MA (US)

(72) Inventors: John R. Bussiere, Littleton, MA (US); James Causey, Simi Valley, CA (US); John D'Arco, Wilmington, MA (US)

(21) Appl. No.: 18/924,403

(22) Filed: Oct. 23, 2024

Related U.S. Application Data
(60) Provisional application No. 63/594,870, filed on Oct. 31, 2023.

(51) Int. Cl. A61M 5/172; A61B 5/145

(52) U.S. Cl. CPC A61M 5/1723; A61B 5/14532

(57) ABSTRACT

Systems for measuring analyte in a user-body of a patient, delivering medicaments to the user-body, and deployment of components thereof are disclosed. The systems include combinations of an analyte sensor, an automated medicament delivery system, a cradle configured to hold the analyte sensor and the automated medicament delivery system on the user-body, and a deployment inserter configured to deploy one or more of the analyte sensor, the automated medicament delivery system, and the cradle onto the user-body.

### CROSS-REFERENCE TO RELATED APPLICATION

This application claims the benefit under 35 U.S.C. § 119(e) of U.S. Provisional Patent Application Ser. No. 63/594,870, filed Oct. 31, 2023, the disclosure of which is hereby incorporated herein in its entirety by this reference.

### TECHNICAL FIELD

The present disclosure generally relates to automated medicament delivery systems and analyte sensors. More particularly, the present disclosure relates to systems and methods for colocation cradles for the automated medicament delivery systems and analyte sensors and deployment thereof.

### BACKGROUND

Analyte sensors and automated medicament delivery systems are used together to obtain analyte data (such as information about glucose, without limitation) related to a user-body (e.g., a body of a patient, human body, without limitation), determine information about amounts of analytes in the user-body (such as a glucose level or concentration, without limitation), generate values that represent amounts of analytes in the user-body (such as glucose values, without limitation), and deliver medicament based at least in part on such analyte levels or values.

Analyte sensors that regularly obtain data related to analyte in the user-body and automated medicament delivery systems that deliver a medicament to the user-body may be adhered to the user-body and a filament or a cannula may be inserted into the user-body. Insertion of a filament or a cannula into the user-body may cause discomfort. Further, changing and positioning multiple devices on the user-body may also cause discomfort. The analyte sensor or automated medicament delivery system may require a support device (e.g., an arm band, etc.) to help keep the analyte sensor or automated medicament delivery system in place.

### BRIEF SUMMARY

In one illustrative embodiment, an assembly is disclosed. The assembly includes an analyte sensor, an automated medicament delivery system, and a cradle. The analyte sensor configured to obtain data about analyte in a user-body. The automated medicament delivery system configured to administer medicament to the user-body. The automated medicament delivery system includes a reservoir configured to hold the medicament therein. The cradle includes a sensor housing and a delivery system mount. The sensor housing configured to at least partially encapsulate the analyte sensor while the cradle is secured to the user-body. The delivery system mount configured to secure the automated medicament delivery system to the cradle.

In another illustrative embodiment, a deployment inserter for a cradle of a delivery and analyte assembly is disclosed. The deployment inserter includes a body structure, a driver plate, a retainer, a main biasing element, a filament sharp, and a cannula sharp. The body structure includes a base defining an opening to an interior of the body structure. The base configured to contact a user-body during deployment of the cradle. The driver plate configured to retain the cradle on a bottom thereof. The cradle positioned closer to the base than the driver plate while in a pre-actuated condition. The retainer secured to the body structure and configured to hold the driver plate in position relative to and within the body structure while in the pre-actuated condition. The main biasing element in a biased condition while in the pre-actuated condition and configured to exert a force on the driver plate while in the pre-actuated condition. The filament sharp configured to insert a filament into the user-body. The cannula sharp configured to insert a cannula into the user-body. The retainer configured to, responsive to an external force, release the driver plate and the force exerted by the main biasing element upon release of the driver plate causes the driver plate to move with the cradle in a first direction toward the base resulting in an adhesive layer of the cradle being adhered to a skin of the user-body, the filament sharp inserting the filament into the user-body, and the cannula sharp inserting the cannula into the user-body.

In another illustrative embodiment, a system is disclosed. The system includes an automated medicament delivery system, a cradle assembly, and a deployment inserter. The automated medicament delivery system configured to administer medicament to a user-body. The automated medicament delivery system includes a reservoir configured to hold the medicament therein. The cradle assembly includes an analyte sensor, and a cradle. The analyte sensor configured to obtain data about analyte in a user-body. The analyte sensor includes a filament. A cradle including a sensor housing configured to secure the analyte sensor to the cradle and position the filament relative to the user-body while the cradle is secured to the user-body, a delivery system mount configured to secure the automated medicament delivery system to the cradle, and an adhesive layer configured to adhere the cradle to the user-body. The deployment inserter includes a body structure, a driver plate, a retainer, a main biasing element, a filament sharp, and a cannula sharp. The body structure includes a base defining an opening to an interior of the body structure. The base configured to contact the user-body during deployment of the cradle assembly. The driver plate with the cradle retained on a bottom thereof. The cradle positioned closer to the base than the driver plate while in a pre-actuated condition of the deployment inserter. The retainer secured to the body structure and configured to hold the driver plate in position relative to and within the body structure while in the pre-actuated condition. The main biasing element in a biased condition while in the pre-actuated condition and configured to exert a force on the driver plate while in the pre-actuated condition. The filament sharp configured to insert the filament into the user-body. The cannula sharp configured to insert the cannula into the user-body. The retainer configured to, responsive to an external force, release the driver plate and the force exerted by the main biasing element upon release of the driver plate causes the driver plate to move with the cradle in a first direction toward the base resulting in the adhesive layer of the cradle being adhered to a skin of the user-body, the filament sharp inserting the filament into the user-body, and the cannula sharp inserting the cannula into the user-body.

In another illustrative embodiment, a method for applying an assembly to a user-body is disclosed. The method includes positioning a deployment inserter over a chosen location to apply a cradle of the assembly thereto with a base of the deployment inserter in contact with a skin of the user-body. The cradle retained on a bottom of a driver plate of the deployment inserter closer to the base. The method also includes causing a retainer of the deployment inserter to release the driver plate. The method further includes, in response to the release of the driver plate, causing the driver plate to press the cradle against the skin of the user-body and to insert a filament and a cannula into the user-body with a main biasing element of the deployment inserter.

In another illustrative embodiment, an apparatus is disclosed. The apparatus includes a base structure, and a hub. The base structure includes a first side configured to sit on a user-body and a second side configured to receive a device to administer medicament to a user-body. The hub formed in the base structure. The hub configured to fluidly couple the first side of the base structure to the second side of the base structure.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1A is an exploded view of a schematic illustration of various embodiments of a delivery and analyte assembly of the present disclosure;

FIG. 1B is a cross-sectional view of the delivery and analyte assembly of FIG. 1A;

FIG. 2 is a perspective view of a deployment inserter for various embodiments of the cradle of FIG. 1A;

FIG. 3A is a perspective view of an exemplary embodiment of a cradle;

FIG. 3B is a top view of the cradle of FIG. 3A;

FIG. 3C is a side view of the cradle of FIG. 3A;

FIG. 4A is a perspective view of an exemplary embodiment of the cradle;

FIG. 4B is a top view of the cradle of FIG. 4A;

FIG. 4C is a side view of the cradle of FIG. 4A;

FIG. 5A is a top view of an exemplary embodiment of a delivery and analyte assembly including the cradle of FIG. 3A;

FIG. 5B is a side view of the delivery and analyte assembly of FIG. 5A;

FIG. 5C is a partial cross-sectional view of the delivery and analyte assembly of FIG. 5A;

FIG. 5D is a schematic diagram illustrating the delivery and analyte assembly in accordance with embodiments;

FIG. 5E is a schematic diagram illustrating the delivery and analyte assembly in accordance with embodiments;

FIG. 6A is a top view of an exemplary embodiment of a delivery and analyte assembly including the cradle of FIG. 4A;

FIG. 6B is a side view of the delivery and analyte assembly of FIG. 6A;

FIG. 6C is a cross-sectional view of the delivery and analyte assembly of FIG. 6A;

FIG. 6D is a bottom view of an automated medicament delivery system of the delivery and analyte assembly of FIG. 6A;

FIG. 7A is a perspective view of a deployment inserter for various embodiments of the cradle;

FIG. 7B is a top cross-sectional view of the deployment inserter of FIG. 7A in a standard condition;

FIG. 7C is a cutaway of the deployment inserter of FIG. 7A taken along the line C-C of FIG. 7B;

FIG. 7D is a cutaway of the deployment inserter of FIG. 7A taken along the line D-D of FIG. 7B;

FIG. 7E is a cross-sectional view of the deployment inserter of FIG. 7A taken along the line C-C of FIG. 7B;

FIG. 7F is a top cross-sectional view of the deployment inserter of FIG. 7A in an actuated condition;

FIG. 7G is a cross-sectional view of the deployment inserter of FIG. 7A shortly after actuation of the actuator;

FIG. 7H is a cross-sectional view of the deployment inserter of FIG. 7A after initial insertion of the filament and the cannula;

FIG. 7I is a cross-sectional view of the deployment inserter of FIG. 7A at full insertion of the filament and the cannula;

FIG. 7J is a cross-sectional view of the deployment inserter of FIG. 7A after full insertion of the filament and the cannula and retraction of the filament sharp and the cannula sharp;

FIG. 7K is a cross-sectional view of the deployment inserter of FIG. 7A after the cradle is deployed on the body of the user;

FIG. 8A is a detailed view of the filament sharp of FIG. 7I prior to release thereof;

FIG. 8B is a detailed view of the filament sharp of FIG. 7J after release thereof;

FIG. 9A is a perspective view of an exemplary embodiment of an assembly of the driver plate and the cradle;

FIG. 9B is an exploded view of the assembly of the driver plate and the cradle of FIG. 9A;

FIG. 10A is a schematic illustration of various embodiments of a fluid path integrity switch in an open condition;

FIG. 10B is a schematic illustration of various embodiments of the fluid path integrity switch of FIG. 10A in a closed condition;

FIG. 11A is a perspective view of an exemplary embodiment of a delivery and analyte assembly of the present disclosure;

FIG. 11B is a top view of the delivery and analyte assembly of FIG. 11A;

FIG. 11C is a schematic illustration of positioning for the delivery and analyte assembly of FIG. 11A;

FIG. 11D illustrates is a perspective view of the delivery and analyte assembly of FIG. 11A including a cover; and

FIG. 11E is a top view of a configuration of the delivery and analyte assembly of FIG. 11A;

FIG. 11F is a top view of a configuration of the cradle of FIG. 11A; and

FIG. 12 is a flowchart of a method for applying a delivery and analyte assembly to the body of a user.

### DETAILED DESCRIPTION

In various embodiments, systems for measuring one or more analytes within the user-body of a patient, delivering medicaments to the user-body, and deployment of components thereof are disclosed. The systems may include any combination of an analyte sensor, an automated medicament delivery system, a cradle configured to hold the analyte sensor and the automated medicament delivery system on the user-body, and a deployment inserter configured to deploy one or more of the analyte sensor, a needle and/or cannula of the automated medicament delivery system, and the cradle onto the user-body.

In the Brief Summary above and in the Detailed Description and the claims below, and in the accompanying drawings, reference is made to particular features (including method acts) of the present disclosure. It is to be understood that the disclosure includes all possible combinations of such particular features. For example, where a particular feature is disclosed in the context of particular embodiments of the delivery and analyte assembly, or a particular claim, that feature can also be used, to the extent possible, in combination with and/or in the context of other particular aspects and embodiments described herein.

As used herein, the term “substantially” in reference to a given parameter, property, or condition means and includes to a degree that one skilled in the art would understand that the given parameter, property, or condition is met with a small degree of variance, such as within acceptable manufacturing tolerances. For example, a parameter that is substantially met may be at least about 90% met, at least about 95% met, or even at least about 99% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

As used herein, the terms “adapted,” “configured,” and “configuration” refers to a size, a shape, a material composition, a material distribution, orientation, and arrangement of at least one feature (e.g., one or more of at least one structure, at least one material, at least one region, at least one device) facilitating use of the at least one feature in a pre-determined way.

As used herein, the term “may” with respect to a material, structure, feature, function, or method act indicates that such is contemplated for use in implementation of an embodiment of the disclosure, and such term is used in preference to the more restrictive term “is” so as to avoid any implication that other compatible materials, structures, features, functions, and methods usable in combination therewith should or must be excluded.

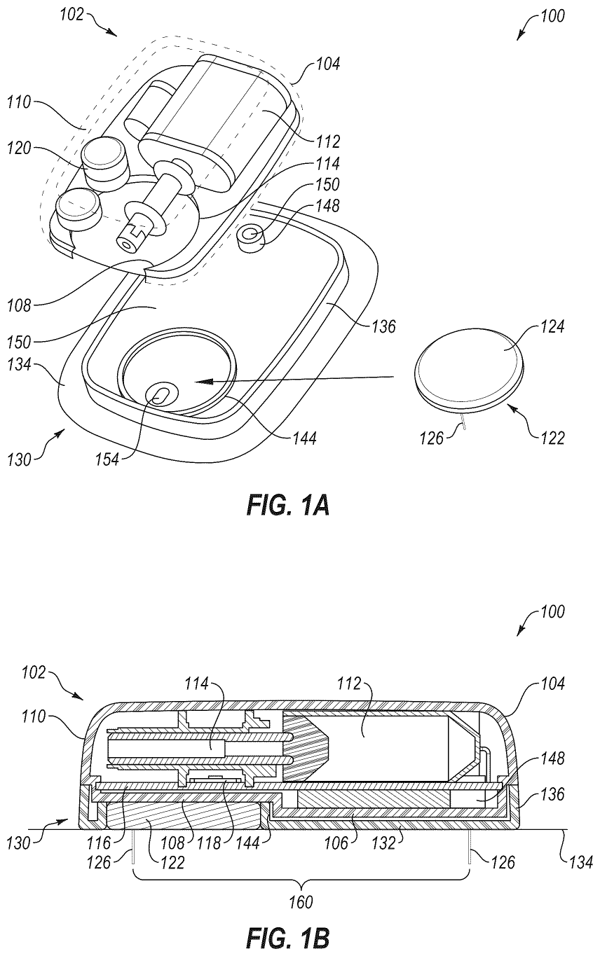

FIG. 1A is an exploded view of a schematic illustration of various embodiments of a delivery and analyte assembly 100 of the present disclosure. FIG. 1B is a cross-sectional view of the delivery and analyte assembly 100 of FIG. 1A. Referring to FIG. 1A and FIG. 1B, the delivery and analyte assembly 100 includes an analyte sensor 122, an automated medicament delivery system 102, and a cradle 130. The analyte sensor 122 is configured to obtain data related to one or more analytes within the user-body of the patient (e.g., measure levels of the one or more analytes directly in the blood or in interstitial fluids of the user-body, without limitation). In various embodiments, the analyte sensor 122 is an analytical bio-sensing device, such as a continuous glucose monitor (CGM) or an interoperable Continuous Glucose Monitor (iCGM) (e.g., the FREESTYLE LIBRE® 3 manufactured by Abbott or the DEXCOM® G6 manufactured by Dexcom, without limitation). The analyte sensor 122 includes a sensor body 124 and a filament 126 extending therefrom. The filament 126 is configured to obtain data related to one or more analytes of the user-body and provide the data to the electronic components of the analyte sensor 122 contained within the sensor body 124. The electronic components may include, inter alia, a processor, memory, and a battery.

The automated medicament delivery system 102 is configured to administer a medicament into the user-body, such as subcutaneously into the user-body. Exemplary medicaments that may be administered by the automated medicament delivery system 102 include insulin, glucagon-like peptide-1 receptor agonist (GLP-1), glucose-dependent insulinotropic polypeptide (GIP), or other hormones, insulin substitutes, and/or combinations of medicaments, such as two or more of insulin, GLP-1, and GIP, or other like hormones. Other medicaments that may be used in other related embodiments include glucagon, morphine, analgesics, fertility medicaments, blood pressure medicaments, chemotherapy drugs, arthritis drugs, weight loss drugs, or the like. Further medicaments may also be delivered by the automated medicament delivery system 102 into the user-body, such as any medicament configured for subcutaneous delivery into the user-body.

In various embodiments, the automated medicament delivery system 102 includes a delivery system housing 104, a reservoir 112, a pump 114, a printed circuit board (PCB) 116, electronic components 118, and one or more power sources 120 (e.g., batteries, without limitation). The delivery system housing 104 includes a delivery system base 106 and a delivery system cap or top housing 110. The delivery system base 106 is configured to support the various components of the automated medicament delivery system 102. In various embodiments, the delivery system base 106 includes a recess 108 sized to receive at least a portion of the analyte sensor 122 therein. The delivery system cap 110 is configured to enclose the various components of the automated medicament delivery system 102 within the delivery system housing 104 and protect the various components.

The reservoir 112 is positioned within the delivery system housing 104 and is configured to store the medicament. The reservoir 112 may be refillable. In various embodiments, the reservoir 112 is positioned opposite/offset from the recess 108.

The pump 114 is configured to cause an amount of the medicament to be administered, via a cannula 150, to the body of the user. The amount may be a bolus, a portion of a bolus, a basal amount, and the like. The pump 114 is in fluid communication with the reservoir 112 and the cannula 150. The pump 114 may include a ratchet gear. In various embodiments, the pump 114 is positioned adjacent to the reservoir 112 and is positioned over the recess 108.

The electronic components 118 may be mounted on the PCB 116 and may be electrically connected to the pump 114, such as via the PCB 116. The electronic components 118 may include one or more processors and memory. The one or more processors may be configured to execute instructions stored on the memory to determine the amount of medicament; cause the pump 114 to deliver the amount to the user-body; and transmit or receive instructions relating to analyte levels or medicament volumes, for example. The one or more power sources 120 may provide electrical energy to the electronic components 118 and the pump 114.

The cradle 130 is a support structure configured for mounting the analyte sensor 122 and the automated medicament delivery system 102 thereto and for securing both the analyte sensor 122 and the automated medicament delivery system 102 to the user-body, such as to the skin of the user-body.

The cradle 130 includes a base structure 132, a sensor housing 144, and a delivery system mount 136. The sensor housing 144 and the delivery system mount 136 are positioned on or otherwise extend from the base structure 132. The base structure 132 includes a first side configured to sit on a user-body and a second side configured to receive the automated medicament delivery system 102 to administer medicament to the user-body.

The sensor housing 144 is configured to position the analyte sensor 122 adjacent to the user-body while the cradle 130 is secured to the user-body with the filament 126 of the analyte sensor 122 inserted within the user-body. In various embodiments, the sensor housing 144 includes a hole formed in the base structure 132 and a flange extending up from the base structure 132. In some of these various embodiments, the sensor housing 144 is configured to secure the analyte sensor 122 to the cradle 130 by an interference fit, a snap fit, a locking feature, threading, an adhesive, bonding, or the like. The hole or perimeter of the sensor housing 144 may be sized and positioned to apply minimal pressure to the analyte sensor 122 via the portion of the delivery system base 106 defining the recess 108. In various embodiments, the pressure is sufficient to securely maintain the analyte sensor 122 in position relative to the user-body, while being less than a pressure that interferes with operation of the analyte sensor 122 (e.g., a force applying too much pressure to the user-body resulting in misreads by the analyte sensor, without limitation).

In some embodiments, the analyte sensor 122 is secured to the user-body prior to the cradle 130 and the base structure 132 may be positioned on the user-body over the analyte sensor 122 after the analyte sensor 122 is placed on the user body (e.g., with an adhesive on the underside of the analyte sensor 122, which adhesive may be part of or separate from adhesive layer 134 and may include a filament opening 154 for the filament to extend therethrough). In such embodiments the sensor housing 144 may comprise a hole around which is a perimeter (the perimeter shown at the end of the lead line for sensor housing 144) which fully or at least partially encompasses, encircles, or is aligned with the analyte sensor 122. In such an embodiment, the analyte sensor 122 may be an off-the-shelf analyte sensor 122. More specifically, the analyte sensor 122 may be purchased separately from the pump 114 and cradle 130.

In various embodiments, the delivery system mount 136 includes a flange extending from the base structure 132. The flange may extend around the base structure 132 and form an edge of the base structure 132. In some of these various embodiments, the delivery system mount 136 is configured to removably secure the automated medicament delivery system 102 to the cradle 130 by an interference fit, a snap fit, a locking feature, or the like. In various embodiments, the base structure 132 includes an indent around an edge thereof that is configured to fit within the delivery system mount 136, while the delivery system cap 110 is configured to include an outer surface that is flush with an outer surface of the delivery system mount 136. In various embodiments, the analyte sensor 122 remains on the user-body longer than the automated medicament delivery system 102 and the automated medicament delivery system 102 is configured to be removable and replaceable by another automated medicament delivery system 102. With the positioning of the sensor housing 144 and the delivery system mount 136, the analyte sensor 122 and the automated medicament delivery system 102 are positioned proximate to one another, which may improve communication therebetween. Communication interference often occurs in other arrangements when the analyte sensor 122 and the automated medicament delivery system 102 are positioned at different locations on the user-body where the user-body may cause such communication interference.

In various embodiments, the cradle 130 also includes a cannula hub 148, a cannula 150, and an adhesive layer 134. The cannula hub 148 is formed in the base structure 132. In various embodiments, the cannula hub 148 includes a cylindrical body at the second side of the base structure 132 that extends away from the first side of the base structure 132, the cannula hub 148 includes a through hole defined by the base structure 132 extending from the first side to the second side and through the cylindrical body. The cannula hub 148 is configured to fluidly couple the first side of the base structure 132 to the second side of the base structure 132. The cannula hub 148 at least partially fluidly couples the cannula 150 to the automated medicament delivery system 102 facilitating medicament held in the reservoir 112 to be delivered to the body via the cannula 150. In particular, the cannula hub 148 is configured to, directly or indirectly, fluidly couple the cannula 150 to the reservoir 112. The cannula hub 148 is configured to form a fluid tight seal with the automated medicament delivery system 102 to prevent leakage of the medicament. In various embodiments, the cannula hub 148 includes a hollow cylinder shape extending from the base structure 132 and may also include a septum and/or an o-ring. The cannula 150 is connected to and fluidly coupled to the cannula hub 148. The cannula 150 extends from the cannula hub 148 through and beyond the base structure 132. While the various embodiments of the cradle 130 include a cannula 150, in other various embodiments, the cannula 150 is included in the automated medicament delivery system 102. More specifically, the automated medicament delivery system 102 may contain therein an insertion mechanism (automated or manual) for the cannula 150. In such case, the cannula hub need only be a hole or hollow cylinder to allow the cannula 150 within the automated medicament delivery system 102 to fire the cannula 150 therethrough and through the skin and into the user's body.

The adhesive layer 134 is joined to the base structure 132 and is configured to adhere the cradle 130 to the user-body. In various embodiments, the adhesive layer 134 is positioned on a bottom of the base structure 132, opposite of the automated medicament delivery system 102, and may extend laterally beyond one or more edges of the base structure 132.

In various embodiments, a distance 160 between a sensor element on the filament 126 and a fluid outlet of the cannula 150 is about one inch or more. In further various embodiments the distance 160 between a sensor element on the filament 126 and a fluid outlet of the cannula 150 is about three inches or more.

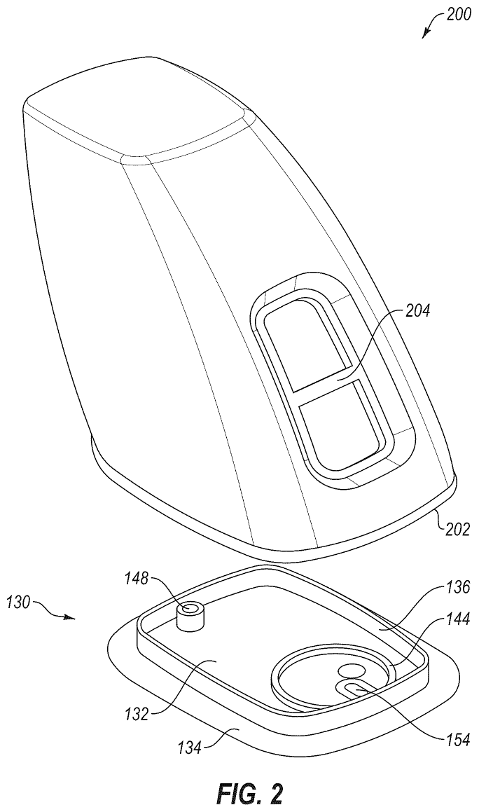

FIG. 2 is a perspective view of a deployment inserter 200 for various embodiments of the cradle 130 of FIG. 1A. The deployment inserter 200 is configured to install the cradle 130 to the body of the user and, in particular, insert the filament 126 and the cannula 150 into the user-body, such as position a tip of the filament 126 and the cannula 150 into a subcutaneous position for obtaining data and administering a medicament, respectively. In various embodiments, the deployment inserter 200 includes an application interface 202 and an actuator 204. The application interface 202 is configured for applying pressure to the adhesive layer 134 to press the adhesive layer 134 against the skin of the user-body and to join the cradle 130 to the skin of the user-body via an adhesive of the adhesive layer 134. The actuator 204 is configured to, once the cradle 130 is in contact with the skin of the user-body, insert the filament 126 and the cannula 150 into the user-body. In various embodiments, the analyte sensor 122 is joined with the cradle 130 prior to the cradle 130 being installed onto the user-body, and the automated medicament delivery system 102 is joined with the cradle 130 after the cradle 130 is installed onto the user-body.

Referring again to FIG. 1A and FIG. 1B, in various embodiments, the automated medicament delivery system 102 includes one or more asymmetric features, such as the recess 108 or cannula hub 148, to ensure proper orientation of the automated medicament delivery system 102 while being joined with the cradle 130.

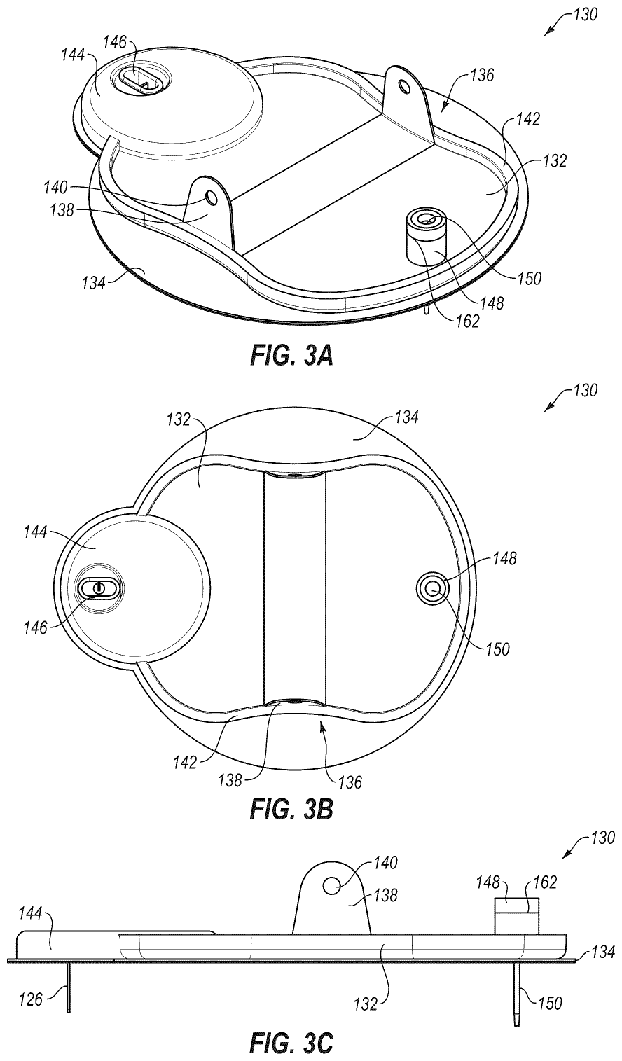

FIG. 3A is a perspective view of an exemplary embodiment of the cradle 130 of FIG. 1A. FIG. 3B is a top view of the cradle 130 of FIG. 3A. FIG. 3C is a side view of the cradle 130 of FIG. 3A. Referring to FIG. 3A-FIG. 3C, in various embodiments, the sensor housing 144 is positioned at least partially inboard and/or at least partially outboard of the delivery system mount 136. Sensor housing 144 may form a housing or encirclement for the components of the analyte sensor 122 (e.g., the components are positioned within the sensor housing 144 without any other housing), an upward facing recess to receive an analyte sensor 122 therein, or a downward facing recess to receive an analyte sensor 122 therein. In embodiments where the sensor housing 144 forms a recess, the sensor housing 144 may include a general shape that matches and is slightly larger than the shape of the analyte sensor 122, or more specifically, may have a concave contour that matches an outer or convex contour of the analyte sensor 122.

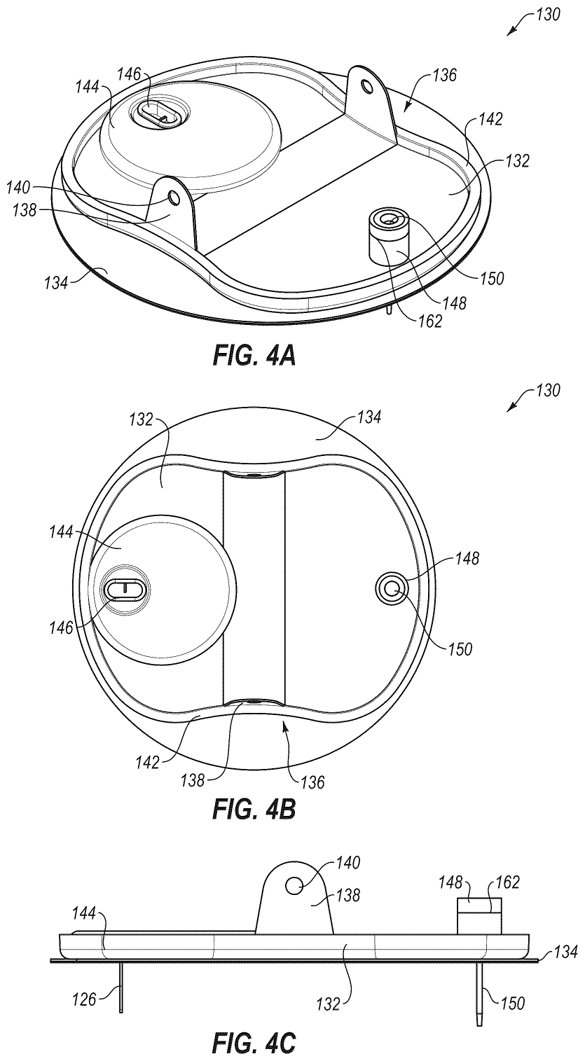

FIG. 4A is a perspective view of an exemplary embodiment of the cradle 130 of FIG. 1A. FIG. 4B is a top view of the cradle 130 of FIG. 4A. FIG. 4C is a side view of the cradle 130 of FIG. 4A. Referring to FIG. 4A-FIG. 4C, in some of these various embodiments, the sensor housing 144 is positioned fully inboard of the delivery system mount 136, or in other words, within a periphery of the cradle 130.

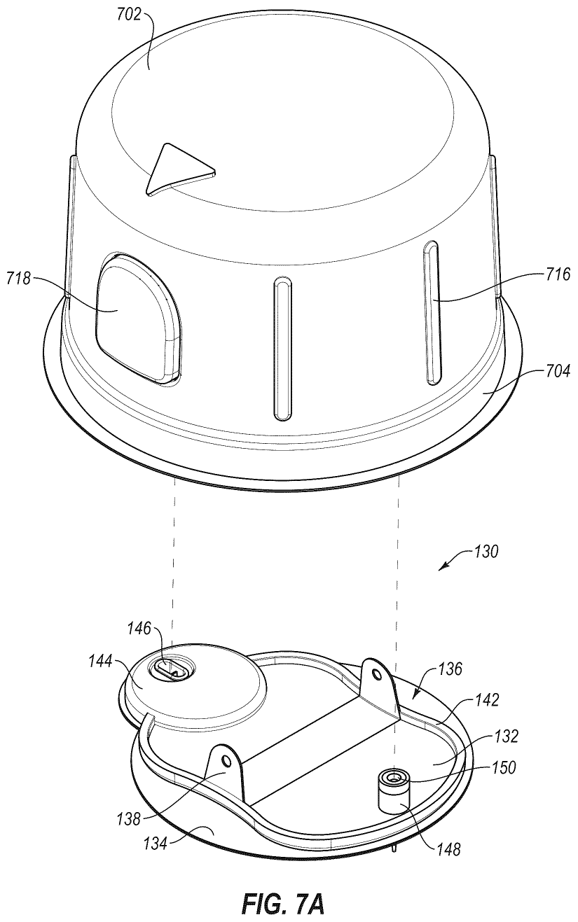

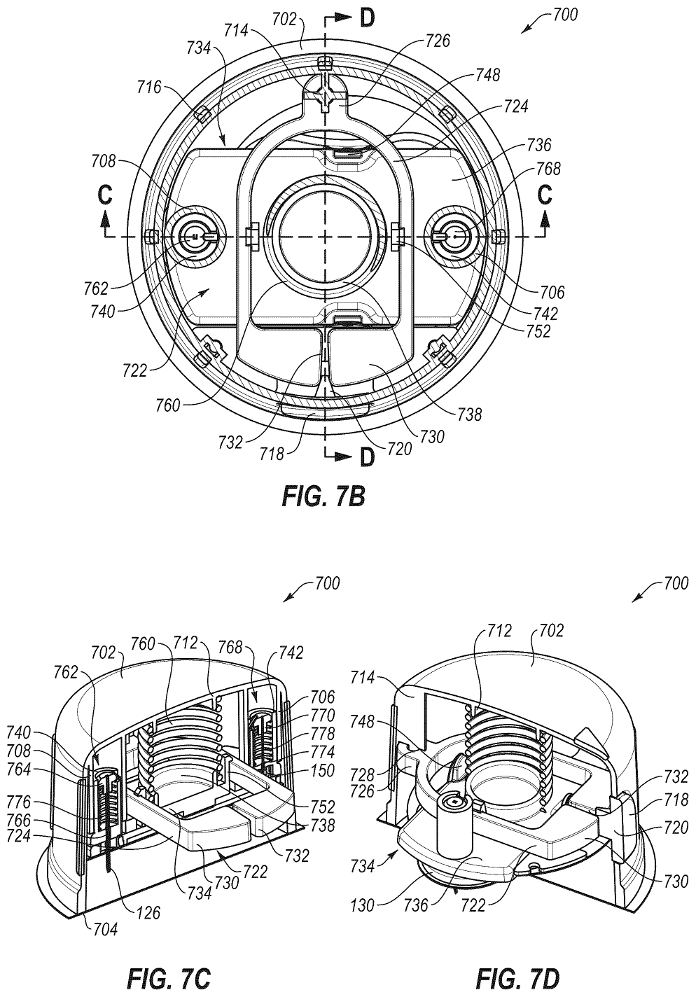

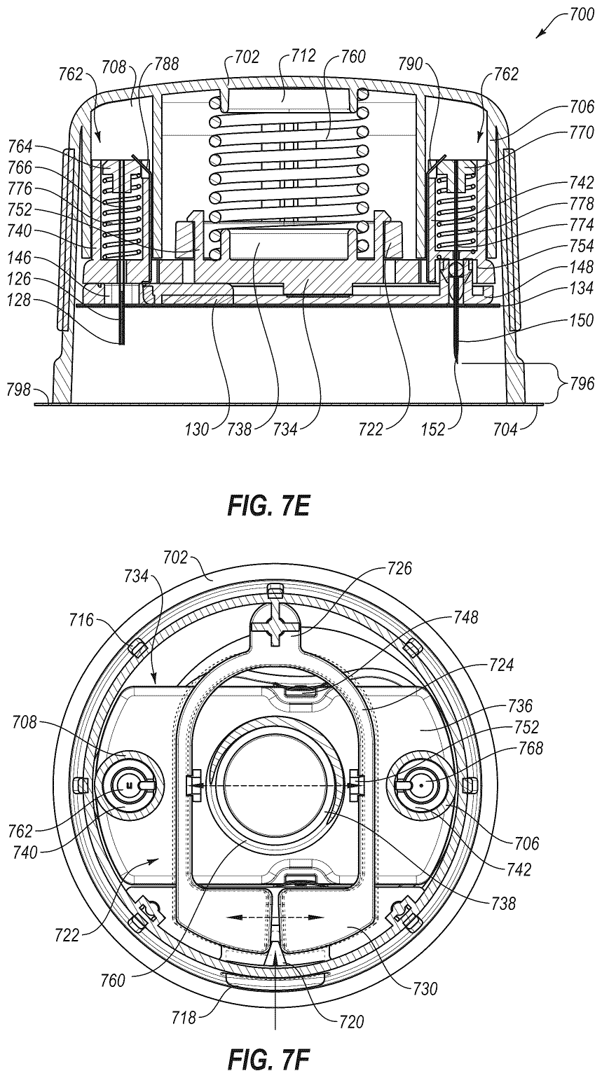

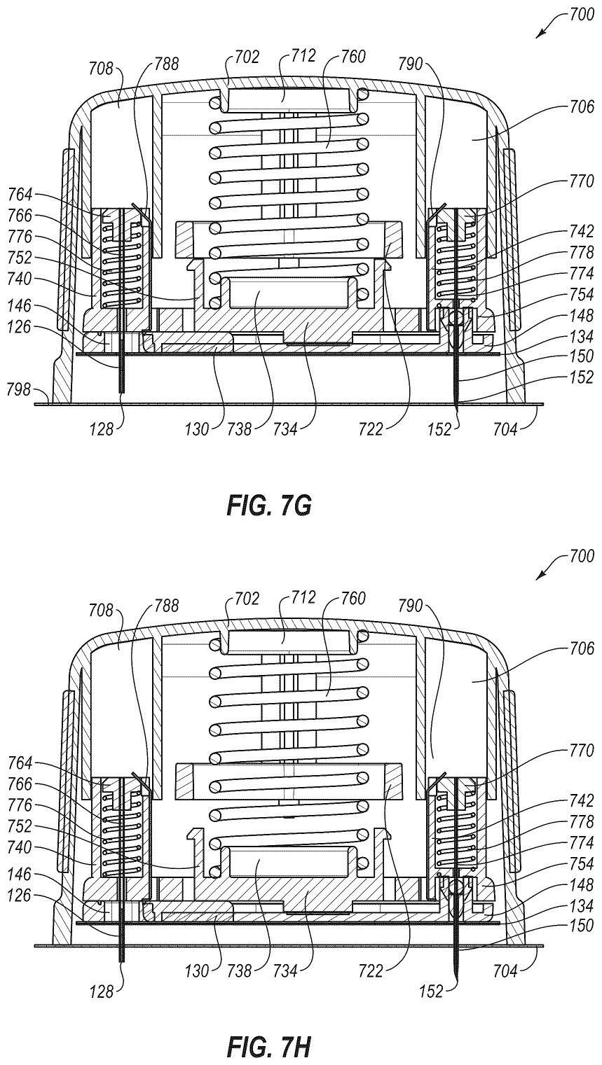

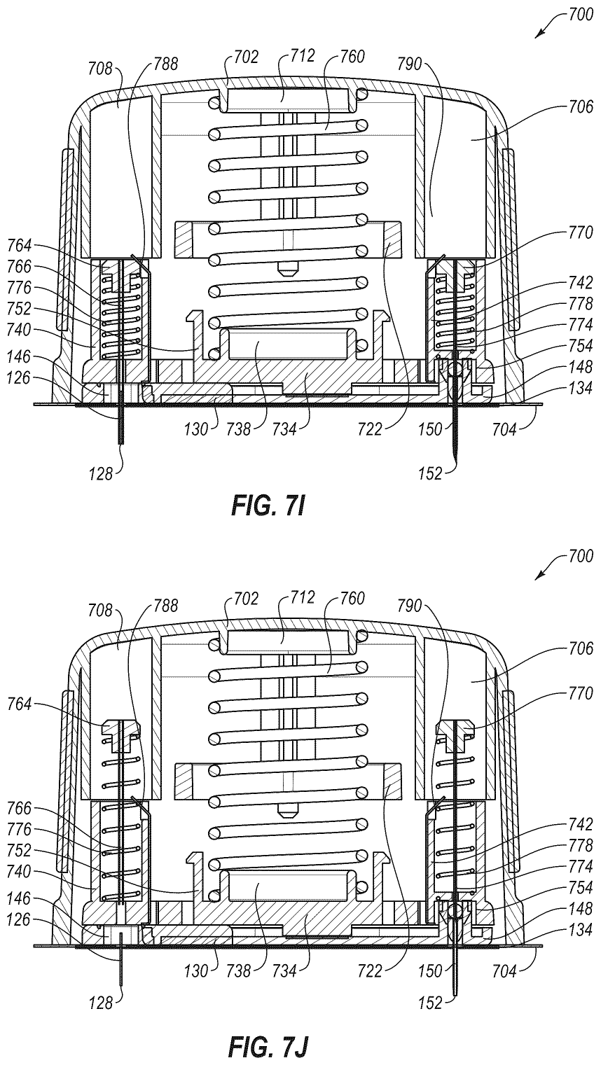

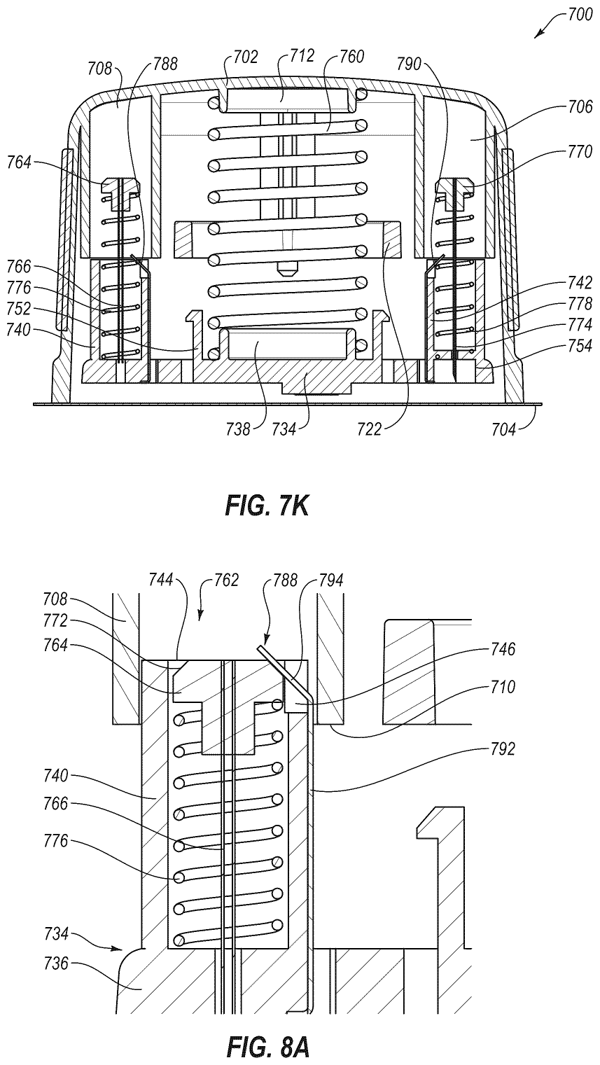

Referring to FIG. 3A-FIG. 4C, the sensor housing 144 includes a sensor opening 146 configured to provide access to the filament 126 to facilitate insertion thereof into the user-body. As will be described in further detail below, the sensor opening 146 is configured to receive at least a portion of a filament sharp 762 (refer to FIG. 7B-FIG. 7K) that is configured to insert the filament 126 into the user-body.

In various embodiments, the cradle 130 includes the filament 126 connected to and extending from the sensor housing 144. In these various embodiments, the sensor housing 144 includes an electronic connector configured to establish a data connection between the filament 126 and the analyte sensor 122.

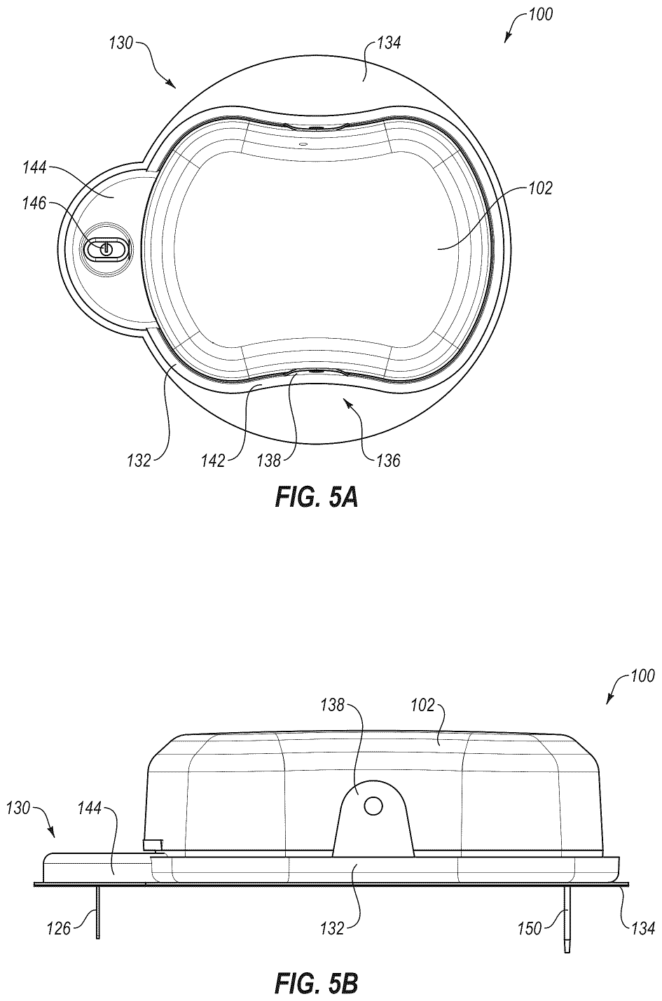

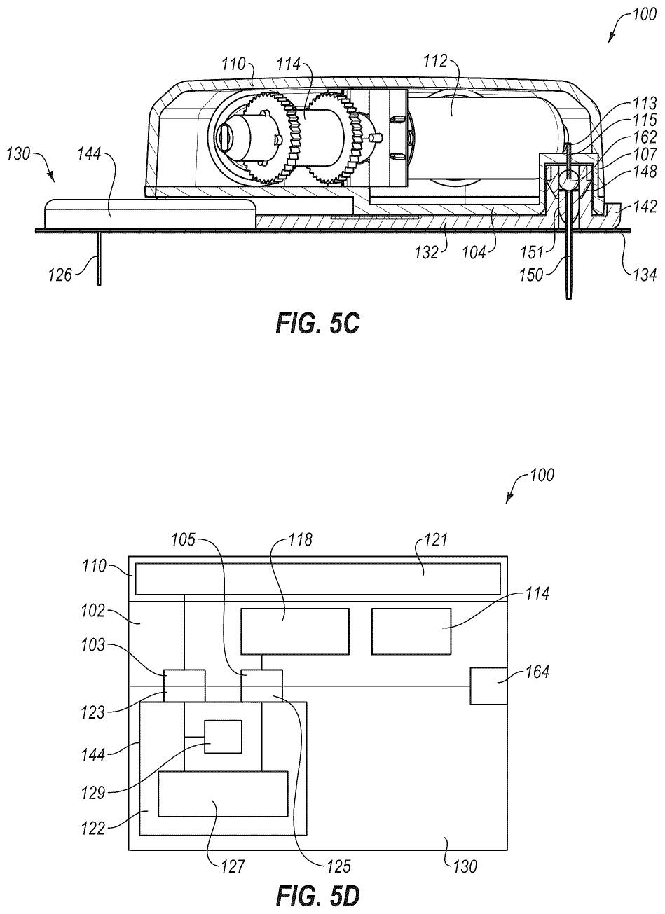

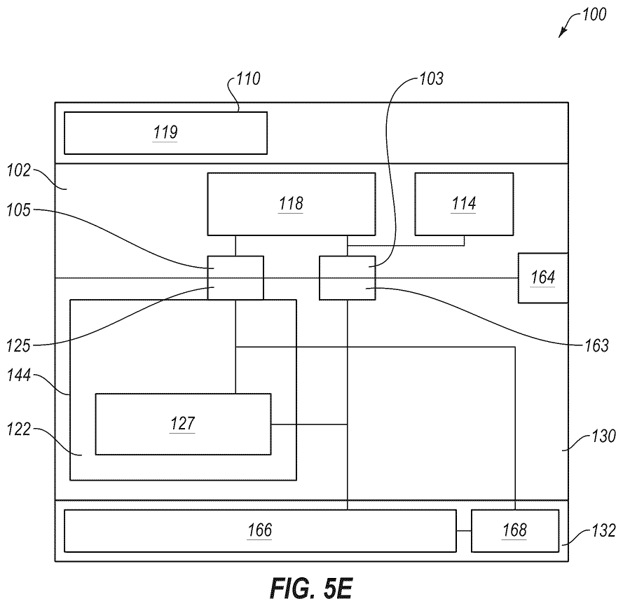

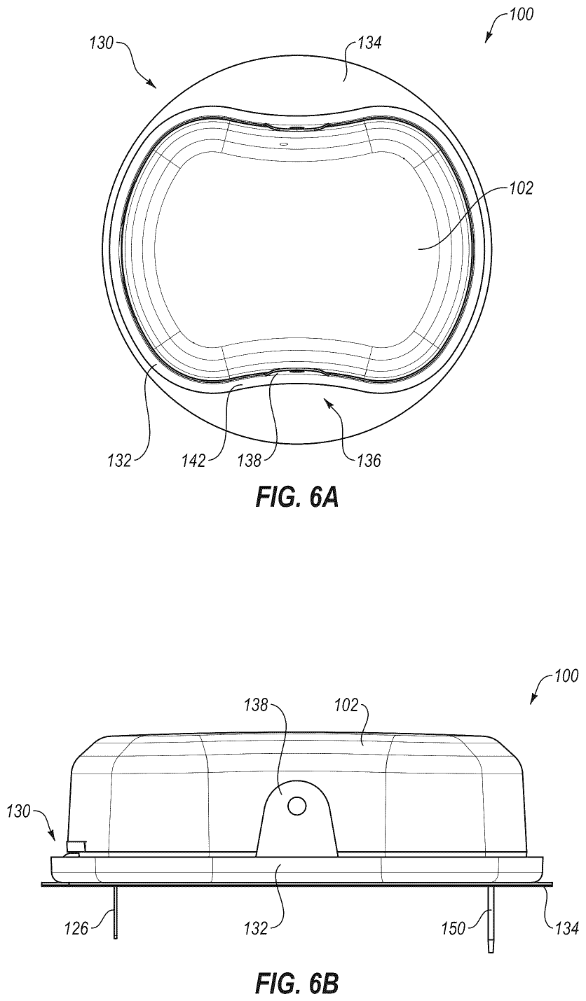

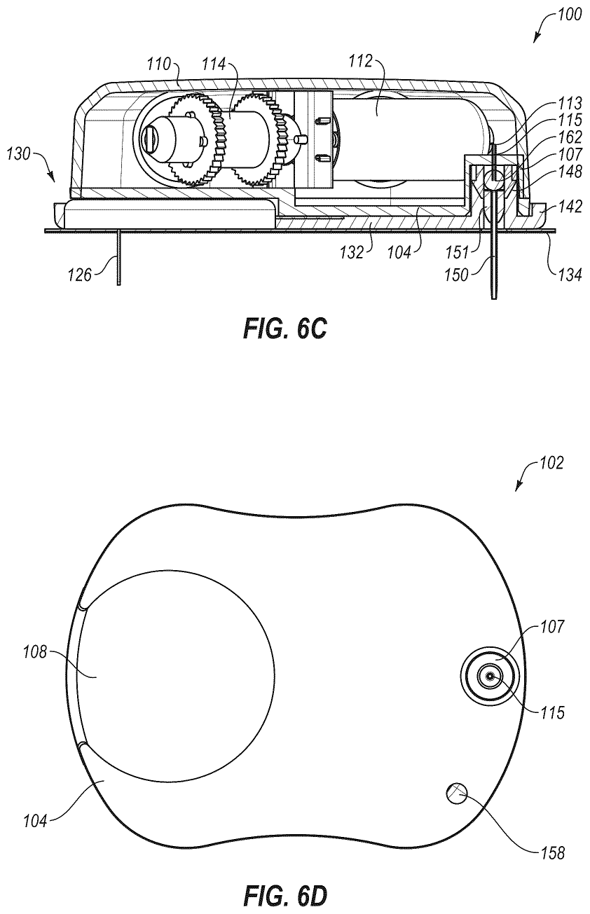

FIG. 5A is a top view of an exemplary embodiment of a delivery and analyte assembly 100 including the cradle 130 of FIG. 3A. FIG. 5B is a side view of the delivery and analyte assembly 100 of FIG. 5A. FIG. 5C is a partial cross-sectional view of the delivery and analyte assembly 100 of FIG. 5A. FIG. 5D is a schematic diagram illustrating the delivery and analyte assembly 100 in accordance with embodiments. FIG. 5E is a schematic diagram illustrating the delivery and analyte assembly in accordance with embodiments. FIG. 6A is a top view of an exemplary embodiment of a delivery and analyte assembly 100 including the cradle of FIG. 4A. FIG. 6B is a side view of the delivery and analyte assembly 100 of FIG. 6A. FIG. 6C is a cross-sectional view of the delivery and analyte assembly 100 of FIG. 6A. FIG. 6D is a bottom view of the automated medicament delivery system 102 of the delivery and analyte assembly 100 of FIG. 6A. Referring to FIG. 3A-FIG. 6D, in various embodiments, the delivery system mount 136 includes a lip 142 extending around and up from at least a majority of the base structure 132 (e.g., excluding a region including an outboard portion of the sensor housing 144 or completely around the base structure 132 with an inboard sensor housing 144). One or more cavities or a continuous cavity, or in some embodiments, one or more protrusions or a continuous protrusion, are proximate the lip 142 are configured to secure the automated medicament delivery system 102 to the cradle 130 by an interference fit, a snap fit, or the like. The lip 142 may include a general shape that matches and is slightly larger than the shape of the automated medicament delivery system 102. The automated medicament delivery system 102, the base structure 132, and the lip 142 may include convex ends and concave sides (e.g., a stadium shape with indented concave sides, without limitation). The concave sides may facilitate the handling of the delivery and analyte assembly 100 and the various components thereof.

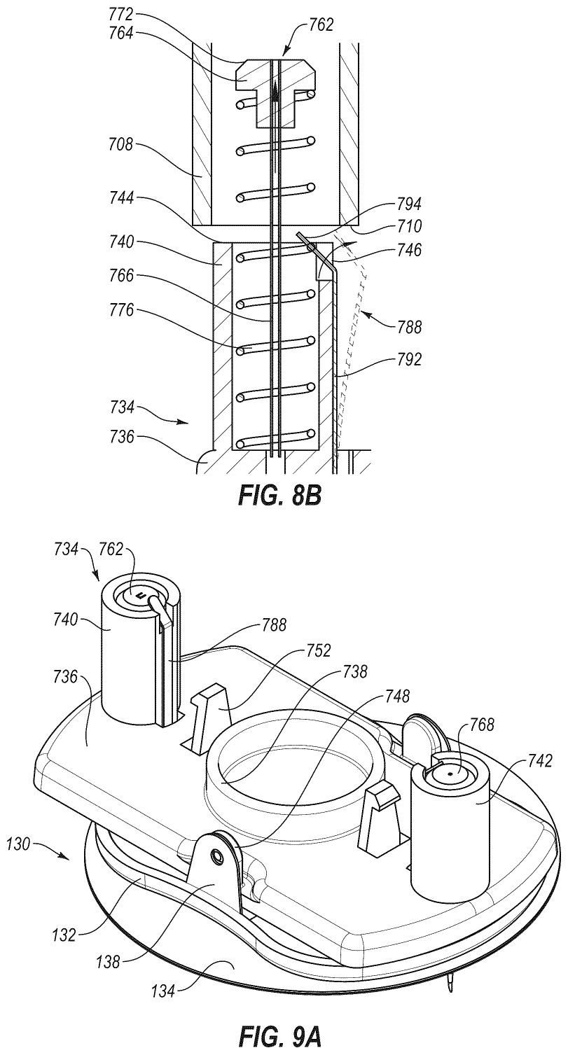

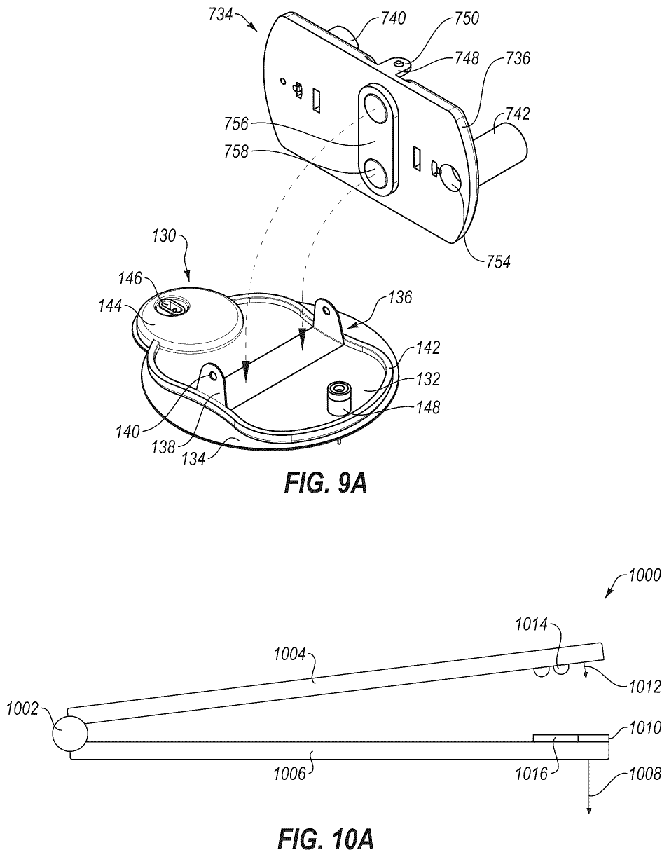

The delivery system mount 136 may also include a bracket 138. The bracket 138 may comprise two parts, one on each side of delivery system mount, or may comprise a single part that extends laterally across the base structure 132 from side to side (e.g., at the narrowest portion of the delivery system mount 136 defined by the minimum of each of the curves defining the concave sides, without limitation). The bracket 138 may be sized to receive the sides of the automated medicament delivery system 102 in an interference or snap condition. The bracket 138 may also include a bracket opening 140 in sides thereof that are configured to receive protruding features of the delivery system cap 110 in a snap configuration. The bracket 138 may include a ferrous material (e.g., a ferromagnetic material, without limitation) configured to be magnetically attracted to one or more magnets positioned on a bottom of the base structure 132. Alternatively, the bracket 138 includes the one or more magnets and the base structure 132 includes the ferrous material configured to be magnetically attracted to the one or more magnets. As will be described in further detail below, the bracket 138 may also be configured for securing the cradle 130 to a deployment inserter 700 (refer to FIG. 7A-FIG. 7K and to FIG. 9A-FIG. 9B).

The cannula hub 148 may include a hollow cylinder shape extending from the base structure 132 with a through hole extending through the base structure 132. In various embodiments, the cannula hub 148 includes one or more seals 162. The one or more seals 162 being positioned at a location chosen from among an outer surface of the cannula hub 148 (e.g., an exterior of the hollow cylinder shape, without limitation, can be seen in FIG. 3A, FIG. 3C, FIG. 4A, and FIG. 4C), an interior/inner surface of the cannula hub 148 (e.g., an interior of the hollow cylinder shape, without limitation, can be seen in FIG. 5C and FIG. 6C), or a combination of the two. The one or more seals 162 are configured to form a fluid tight seal between the cannula hub 148 and the automated medicament delivery system 102.

In various embodiments, the fluid tight seal is formed between the cannula hub 148 and the automated medicament delivery system 102 while forming a fluidic connection between the reservoir 112 and the cannula 150. In some of these embodiments, the automated medicament delivery system 102 includes a hub receiver 107, a mating needle 115, and a conduit 113. The hub receiver 107 extends up from a base of the delivery system housing 104 and defines a cavity configured to receive the cannula hub 148. The cavity defined by the hub receiver 107 is sized to receive the cannula hub 148 therein. The conduit 113 fluidly couples the mating needle 115 to the reservoir 112. The mating needle 115 extends from an end of the conduit 113 into the cavity defined by the hub receiver 107. While the cradle 130 and the automated medicament delivery system 102 are mated together, the mating needle 115 extends into the cannula hub 148 and through a seal of the one or more seals 162. The seal is positioned within the cannula hub 148 and the mating needle 115 extending therethrough forms a fluidic connection with the cannula 150. In these embodiments, the mating needle 115 punctures through the seal within the cannula hub 148 during the mating process between the cradle 130 and the automated medicament delivery system 102. The one or more seals 162 may be a viscoelastic material (e.g., silicon rubber, without limitation). The seal within the cannula hub 148 may be a solid object that seals with an inner surface of the cannula hub 148 (e.g., a sphere, a cylinder, a septum, without limitation).

An end of the cannula 150 is connected to the base structure 132 within the cannula hub 148. In various embodiments, the cradle 130 includes a cannula support 151 at the end of the cannula 150. The cannula support 151 may be connected to the end of the cannula 150 (e.g., welded thereto, without limitation) or may be integrally formed with the cannula 150 as a single object. An exterior of the cannula support 151 is joined to an inner surface of the cannula hub 148 (e.g., welded thereto, without limitation). The cannula support 151 may be positioned within the cannula hub 148 adjacent to the seal positioned within the cannula hub 148 closer to the first side of the base structure 132 relative to the seal.

In various embodiments, the automated medicament delivery system 102 includes a fill port 158 in fluid communication with the reservoir 112 configured for supplying the medicament to the reservoir 112. In the embodiments illustrated in FIG. 6D, the fill port 158 is positioned and formed in the delivery system housing 104 with an outlet thereof in a bottom of the delivery system housing 104. The fill port 158 may be covered by the cradle 130 while the automated medicament delivery system 102 is secured thereto.

In various embodiments, the cradle 130 includes one or more of: wires, connectors, and a PCB configured to form a physical data connection between the automated medicament delivery system 102 and the analyte sensor 122. As noted above, the cradle 130 may include an analyte sensor 122 integrated therein with the various components of an analyte sensor 122 positioned within the sensor housing 144. In some of these various embodiments, only one of the automated medicament delivery system 102, analyte sensor 122, and the cradle 130 includes a wireless radio for communicating with external devices, such as a controller or a mobile device for communicating with and controlling the automated medicament delivery system 102 and the analyte sensor 122. The wireless radio may be configured for a wireless communication protocol, such as according to a WI-FI®, BLUETOOTH®, near-field communication, cellular, or any other radio frequency, infrared, or optical communication technology. The cradle in any of the base structure 132, the sensor housing 144, and the delivery system mount 136, which may strengthen wireless communication by the automated medicament delivery system 102 and/or the analyte sensor 122.

In various embodiments, the automated medicament delivery system 102 is configured to provide a primary power source 121 for the delivery and analyte assembly 100. The primary power source 121 may be integrated into part of delivery system cap or top housing 110 (as illustrated in FIG. 5D), and/or there may be the one or more power sources 120 positioned within the automated medicament delivery system 102 (e.g., within delivery system housing 104 or mounted to the base structure 132, without limitation). The primary power source 121 may be configured to supply power for operation of both the automated medicament delivery system 102 and the analyte sensor 122 while in use on the user-body.

In some of these various embodiments, the automated medicament delivery system 102 includes a delivery system power contact 103 and analyte sensor 122 or the cradle 130 includes a sensor power contact 123. The delivery system power contact 103 and the sensor power contact 123 are configured to electronically couple to transfer energy from the primary power source 121 to sensor electronics 127 while the automated medicament delivery system 102 is connected to the cradle 130. The delivery system power contact 103 and the sensor power contact 123 may include any type of structure that facilitates the electrical connection (e.g., connection pads, without limitation). The delivery system power contact 103 may be integrated into the base structure 132 of the automated medicament delivery system 102, and the sensor power contact 123 may be integrated into the sensor housing 144. The analyte sensor 122 may include a temporary power source 129 (as illustrated in FIG. 5D) that is configured to provide sufficient power to the sensor electronics 127 while the automated medicament delivery system 102 is disconnected from the cradle 130. The temporary power source 129 may comprise a supercapacitor, a capacitor, or a battery.

In various embodiments, the automated medicament delivery system 102 includes a delivery system electronics contact 105 and analyte sensor 122 or the cradle 130 includes a sensor electronics contact 125 (as illustrated in FIGS. 5D and 5E). The delivery system electronics contact 105 and the sensor electronics contact 125 are configured to electrically couple the electronic components 118 of the automated medicament delivery system 102 to the sensor electronics 127 while the automated medicament delivery system 102 is connected to the cradle 130. The delivery system electronics contact 105 and the sensor electronics contact 125 may include any type of structure that facilitates the electrical connection (e.g., connection pads, without limitation). The delivery system electronics contact 105 may be integrated into the base structure 132 of the automated medicament delivery system 102, and the sensor electronics contact 125 may be integrated into the sensor housing 144.

In various embodiments, the cradle 130 is configured to provide a primary power source 166 for the delivery and analyte assembly 100. The primary power source 166 may be integrated into the base structure 132 (as illustrated in FIG. 5E). The primary power source 166 is configured to supply power for operation of both the automated medicament delivery system 102 and the analyte sensor 122 while in use on the user-body. The automated medicament delivery system 102 may include a temporary power source 119 that is configured to provide sufficient power to the electronics 118 while the automated medicament delivery system 102 is disconnected from the power source 166. The temporary power source 119 may comprise a supercapacitor, a capacitor, or a battery.

In some of these various embodiments, the automated medicament delivery system 102 includes a delivery system power contact 103 and the cradle 130 includes a cradle power contact 163. The delivery system power contact 103 and the cradle power contact 163 are configured to electronically couple to transfer energy from the primary power source 166 to the electronic components 118 and the pump 114 of the automated medicament delivery system 102 while the automated medicament delivery system 102 is connected to the cradle 130.

In various embodiments, the cradle includes one or more sensors 168. The one or more sensors 168 may be powered by the primary power source 166 and may electronically connected with the sensor electronics 127, the sensor electronics contact 125, or other electronics mounted on the cradle 130. The one or more sensors 168 may include an on-body sensor and/or a motion sensor. The on-body sensor is configured to detect whether the cradle 130 is attached to the user-body. The motion sensor may include at least one of an accelerometer, a gyroscope, and an inertial measurement units (IMU) or similar sensors and may detect motion of the user body.

The cradle 130 and/or the automated medicament delivery system 102 may include a release mechanism 164 or other features configured to facilitate the removal of the automated medicament delivery system 102 for replacement thereof. In various embodiments, the release mechanism 164 is configured to release the automated medicament delivery system 102. Release may occur by pressing the release mechanism, or in some embodiments the automated medicament delivery system 102, toward the cradle 130.

In various embodiments, one or more of the automated medicament delivery system 102, the analyte sensor 122, and the cradle 130 includes a Radio-Frequency Identification (RFID) device, such as a RFID tag or RFID reader, a Near-Field Communication (NFC) sensor, and/or BLUETOOTH®. The RFID device, NFC sensor, and/or BLUETOOTH® may identify the automated medicament delivery system 102, analyte sensor 122, and/or cradle 130 that are currently in use/being connected along with various details associated therewith, type of device, manufacturer, and the like. Further, the NFC sensor may be used for waking up or activating the automated medicament delivery system 102 and/or the analyte sensor 122. The NFC sensor may also be used to synchronize the automated medicament delivery system 102 or the analyte sensor 122 with a controller or mobile device.

FIG. 7A is a perspective view of a deployment inserter 700 for various embodiments of the cradle. FIG. 7B is a top cross-sectional view of the deployment inserter 700 of FIG. 7A in a standard condition. FIG. 7C is a cutaway of the deployment inserter 700 of FIG. 7A taken along the line C-C of FIG. 7B. FIG. 7D is a cutaway of the deployment inserter 700 of FIG. 7A taken along the line D-D of FIG. 7B. FIG. 7E is a cross-sectional view of the deployment inserter 700 of FIG. 7A taken along the line C-C of FIG. 7B. Referring to FIG. 7A-FIG. 7E, the deployment inserter 700 is configured to receive the cradle 130 and upon actuation, press the cradle 130 against the skin of the user-body to secure the cradle 130 thereto via the adhesive layer 134 and to insert the filament 126 and the cannula 150 into the user-body.

In various embodiments, the deployment inserter 700 includes a body structure 702, an actuator 718, a retainer 722, a driver plate 734, a main biasing element 760, a filament sharp 762, a cannula sharp 768, a filament biasing element 776, and a cannula biasing element 778. The body structure 702 includes a base 704 defining an opening to an interior of the body structure 702 for receiving the cradle 130 and securing the cradle 130 to the user-body. The base 704 may be configured to contact the user-body/skin during deployment of the cradle 130. In particular, a surface of the base 704 positioned at a bottom of the body structure 702 is configured to contact the user-body/skin. The body structure 702 may include a hollow cylinder shape with a closed top opposite the base 704. The body structure 702 may include gripping features 716 protruding from the hollow cylinder shape. The gripping features 716 may include dissimilar materials than the remainder of the body structure 702. The body structure 702 also includes a retainer mount 714 configured for mounting the retainer 722 to the body structure 702. The retainer mount 714 may protrude from a sidewall of the body structure 702.

The body structure 702 also includes a body biasing mount 712 configured to hold an end of the main biasing element 760 in position relative to the body structure 702. In various embodiments, the body biasing mount 712 extends from the closed end of the body structure 702 in a first direction toward the base 704. In some of these various embodiments, the body biasing mount 712 includes a cylindrical shape, and in various embodiments, includes a hollow cylinder shape. The body biasing mount 712 is configured to receive an end of the main biasing element 760 at one of a radially outward and a radially inward surface to hold the main biasing element 760 in position relative to the body structure 702. In various other embodiments, the body biasing mount 712 includes a recess formed in the body structure 702 that is configured to receive the end of the main biasing element 760 therein.

The body structure 702 also includes a first actuation guide 706 and a second actuation guide 708 extending from the closed end of the body structure 702 toward the base 704. The first actuation guide 706 and the second actuation guide 708 are positioned offset from the body biasing mount 712 (e.g., each positioned adjacent to the sidewall of the body structure 702 and offset approximately 90 degrees in opposite directions from the body biasing mount 712, without limitation).

The actuator 718 is configured to cause the retainer 722 to release the driver plate 734. In various embodiments, the actuator 718 includes an actuator wedge 720 extending inward toward a middle of the interior of the body structure 702.

The retainer 722 is configured to hold the driver plate 734 in position relative to and within the body structure 702 in a standard, pre-actuated, condition. The retainer 722 is also configured to be actuated to release the driver plate 734 in response to actuation of the actuator 204. The retainer 722 includes a retainer body 724, a retainer connector 726 formed by the retainer body 724, and a retainer base 730. The retainer connector 726 is configured to connect the retainer 722 to the body structure 702 and, in particular, to the retainer mount 714 of the body structure 702. The retainer connector 726 may include a connector opening 728 formed in the retainer body 724 that receives a portion of the retainer mount 714.

The retainer base 730 is positioned opposite the retainer connector 726 and is configured to cause the retainer 722 to release the driver plate 734 upon actuation of the actuator 718. In various embodiments, the retainer 722 includes a wishbone shape with the retainer connector 726 at a closed end of the wishbone shape and the retainer base 730 defines a retainer opening 732 opposite the retainer connector 726. However, other shapes and configurations of the retainer 722 are also contemplated. The retainer opening 732 is configured to receive a tip of the actuator wedge 720.

The driver plate 734 is positioned within the body structure 702 and is supported by the retainer 722 while in the standard, pre-actuated, condition. As will be described in greater detail below, the driver plate 734 retains the cradle 130 on a bottom thereof, the bottom being closer to the base 704 than the closed end of the body structure 702.

In various embodiments, the driver plate 734 includes a plate body 736, a plate biasing mount 738, one or more retention clips 752, a filament sharp guide 740, and a cannula sharp guide 742. The plate body 736 generally includes a flat and thin shape (e.g., thin relative to a length and width thereof). The plate body 736 includes a hub receiving hole 754 configured to receive at least a portion of the cannula hub 148 therein.

The plate biasing mount 738 extends from a top of the plate body 736 in a second direction (opposite the first direction) toward the body biasing mount 712 while the driver plate 734 is positioned within the body structure 702 (e.g., in a direction opposite the bottom that supports the cradle 130 and toward a closed end of the body structure 702). The plate biasing mount 738 includes a cylindrical shape, and in various embodiments, includes a hollow cylinder shape. The plate biasing mount 738 is configured to receive an end of the main biasing element 760 at one of a radially outward and a radially inward surface to hold the main biasing element 760 in position relative to the plate biasing mount 738, the end being opposite the end received by the body biasing mount 712.

The one or more retention clips 752 are configured to secure the driver plate 734 to the retainer 722 while the deployment inserter 200 is in the standard, pre-actuated, condition. Upon actuation of the actuator 718 the one or more retention clips 752 are configured to be released from the retainer 722, allowing a force exerted by the main biasing element 760 to cause the driver plate 734 to move in the first direction. In various embodiments, each of the one or more retention clips 752 extends upwards from the plate body 736 in the second direction and includes a protrusion extending outward, relative to a middle of the driver plate 734/body structure 702. The protrusion is configured to overlap with a respective side of the retainer body 724 to prevent movement of the driver plate 734 relative to the retainer 722. The protrusion may include a wedge shape to facilitate assembly of the driver plate 734, the wedge shape configured to cause respective ones of the one or more retention clips 752 to deflect temporarily inward to pass the respective side of the retainer body 724 until the driver plate 734 is fully inserted within the body structure 702.

In various embodiments, the retainer 722 includes the one or more retention clips 752, which are configured to assemble to a recess formed within the plate body 736 until the actuator 718 is actuated.

The filament sharp guide 740 is configured to position the filament sharp 762 relative to the driver plate 734. The filament sharp 762 including a filament sharp head 764 and a filament needle 766. The filament needle 766 is configured to insert the filament 126 into the user-body. The filament sharp guide 740 is also configured to hold the filament biasing element 776 in a biased (e.g., compressed) condition between the plate body 736 and the filament sharp head 764. In various embodiments, the filament sharp guide 740 extends upward from the plate body 736, in the second direction and includes a hollow cylinder shape with an outer radius configured to be received within the second actuation guide 708 and an inner radius configured to receive the filament sharp head 764.

The cannula sharp guide 742 is configured to position the cannula sharp 768 relative to the driver plate 734. The cannula sharp 768 including a cannula sharp head 770 and a cannula needle 774. The cannula needle 774 is configured to insert the cannula 150 into the user-body. The cannula sharp guide 742 is also configured to hold the cannula biasing element 778 in a biased (e.g., compressed) condition between the plate body 736 and the cannula sharp head 770. In various embodiments, the cannula sharp guide 742 extends upward from the plate body 736, in the second direction and includes a hollow cylinder shape with an outer radius configured to be received within the first actuation guide 706 and an inner radius configured to receive the cannula sharp head 770.

The main biasing element 760 is in a biased condition (e.g., compressed, without limitation) between the closed end of the body structure 702 and the driver plate 734 while the deployment inserter 700 is in the standard, pre-actuated, condition. The main biasing element 760, the filament biasing element 776, and the cannula biasing element 778 may each include a spring (e.g., a coil spring or leaf spring, without limitation) or other compressible materials that applies a force while compressed sufficient to move one or more elements of the deployment inserter 700 (e.g., the main biasing element 760 applies a force to the driver plate 734, and upon actuation of the actuator 718 and release of the driver plate 734/the one or more retention clips 752, expands and causes the driver plate 734 and the cradle 130 to move in the first direction toward the body of the user).

In various embodiments, a cannula tip 152 is offset from the base 704 by a minimum clearance 796 while the driver plate 734 is in the pre-actuated condition. In various embodiments, the minimum clearance 796 is about 0.25 inches (refer to FIG. 7E).

In various embodiments, the deployment inserter 700 includes a seal 798 positioned over the opening defined by the base 704 and configured to maintain a sterile environment within the body structure 702. Prior to application of the cradle 130 to the user-body and actuation of the actuator 718, the seal 798 is removed.

In various embodiments, the deployment inserter 700 is configured to insert each of the filament 126 and the cannula 150 into the user-body at about the same time (e.g., within 100 milliseconds, without limitation).

In various embodiments, the deployment inserter 700 and the delivery and analyte assembly 100 are provided as a system to the patient with the analyte sensor 122 mounted in the cradle 130 as a cradle assembly and the cradle assembly positioned within the deployment inserter 700 (e.g., the deployment inserter 700 with the cradle assembly is sterilized with the seal 798 maintaining the sterile environment therein, without limitation) and the automated medicament delivery system 102 provided therewith (e.g., without being mounted to the cradle 130) and configured to be mounted to the cradle 130 after the cradle assembly is deployed onto the user-body.

FIGS. 7F-7K illustrate the actuation of the deployment inserter 700 and application of the cradle 130 to the body of a user thereby. FIG. 7F is a top cross-sectional view of the deployment inserter 700 of FIG. 7A in an actuated condition. FIG. 7G is a cross-sectional view of the deployment inserter 700 of FIG. 7A shortly after actuation of the actuator 718. FIG. 7H is a cross-sectional view of the deployment inserter 700 of FIG. 7A after initial insertion of the filament 126 and the cannula 150. FIG. 7I is a cross-sectional view of the deployment inserter 700 of FIG. 7A at full insertion of the filament 126 and the cannula 150. FIG. 7J is a cross-sectional view of the deployment inserter 700 of FIG. 7A after full insertion of the filament 126 and the cannula 150 and retraction of the filament sharp 762 and the cannula sharp 768. FIG. 7K is a cross-sectional view of the deployment inserter 700 of FIG. 7A after the cradle 130 is deployed on the body of the user. Referring to FIGS. 7F-7K, responsive to an external force, the driver plate 734 is released. In various embodiments, actuation of the actuator 718 applies the external force, directly or indirectly, initiating the driver plate 734 to release from the retainer 722. In some of these various embodiments, the actuator 718 is configured to move inward (refer to FIG. 7F). The inward movement of the actuator 718 causes the actuator wedge 720 to move further into the retainer opening 732 applying the external force to the retainer base 730 resulting in each side of the retainer base 730 being separated and the sides of the retainer body 724 moving further apart. The movement is sufficient to release the one or more retention clips 752 (e.g., the protrusion of each of the one or more retention clips 752 no longer overlapping with the retainer body 724, without limitation).

Upon release of the one or more retention clips 752, the main biasing element 760 pushes the driver plate 734 in the first direction, toward the user-body (refer to FIG. 7G-FIG. 7I). The movement of the driver plate 734 causes the cradle 130 to contact the skin of the user-body, causing the adhesive layer 134 to adhere thereto, and causes the filament sharp 762 to insert the filament 126 into the user-body and the cannula sharp 768 to insert the cannula 150 into the user-body (e.g., positioning at least the filament tip 128 and the cannula tip 152 subcutaneously within the user-body, without limitation).

Upon insertion of the filament 126 and the cannula 150, the deployment inserter 700 is configured to withdraw the filament sharp 762 and the cannula sharp 768 from the user-body (refer to FIG. 7J).

FIG. 8A is a detailed view of the filament sharp 762 of FIG. 7I prior to release thereof. FIG. 8B is a detailed view of the filament sharp 762 of FIG. 7J after release thereof. Referring to FIG. 7J, FIG. 8A, and FIG. 8B, in various embodiments, the driver plate 734 includes a filament sharp lock 788 configured to retain the filament sharp 762 within the filament sharp guide 740 and a cannula sharp lock 790 configured to retain the cannula sharp 768 within the cannula sharp guide 742 until insertion of the filament 126 and the cannula 150 into the user-body. The filament biasing element 776 is configured to withdraw the filament sharp 762 from the user-body upon release of the filament sharp lock 788, and the cannula biasing element 778 is configured to withdraw the cannula sharp 768 from the user-body upon release of the cannula sharp lock 790.

In some of these various embodiments, the filament sharp lock 788 and the cannula sharp lock 790 include a lock body 792 and a lock arm 794. The lock body 792 is secured to the plate body 736 and extends along a respective filament sharp guide 740/cannula sharp guide 742. The lock arm 794 extends from a distal end of the lock body 792 over a respective filament sharp head 764/cannula sharp head 770. Each of the filament sharp guide 740 and the cannula sharp guide 742 may include a sharp guide opening 746 configured for the lock arm 794 to pass therethrough and extend over the respective filament sharp head 764/cannula sharp head 770. In the embodiments illustrated, the sharp guide opening 746 extends from a sharp guide end 744 toward the plate body 736.

The lock body 792 of the filament sharp lock 788 is positioned between the second actuation guide 708 and the filament sharp guide 740 and the lock body 792 of the cannula sharp lock 790 is positioned between the first actuation guide 706 and the cannula sharp guide 742 while the deployment inserter 700 is in the standard, pre-actuated, condition and until the cradle 130 is substantially deployed, such as about fully deployed. Upon insertion of the filament 126 and the cannula 150, the lock body 792 is released allowing the respective filament biasing element 776/cannula biasing element 778 to push the respective filament sharp head 764/cannula sharp head 770 in the second direction, pushing the lock arm 794 out from over the sharp guide end 744 and withdrawing the respective filament sharp 762/cannula sharp 768 from the user-body. A length of the filament sharp guide 740 and the cannula sharp guide 742 may be configured such that the respective sharp guide openings 746 therein exit an actuation guide end 710 of the respective first actuation guide 706/second actuation guide 708 at or prior to the cradle 130 pressing against the skin of the user. The sharp guide end 744 may exit the actuation guide end 710 at or prior to the cradle 130 pressing against the skin of the user.

The lock arm 794 may extend from the lock body 792 between 90 degrees and 180 degrees. The filament sharp head 764 and the cannula sharp head 770 may each include a head chamfer 772 at a top surface thereof, opposite the filament needle 766/cannula needle 774. The head chamfer 772 may be angled to align with the angle of the lock arm 794.

FIG. 9A is a perspective view of an exemplary embodiment of an assembly of the driver plate 734 and the cradle 130. FIG. 9B is an exploded view of the assembly of the driver plate 734 and the cradle 130 of FIG. 9A. The driver plate 734 and the cradle 130 are configured to couple together and remain coupled until the cradle 130 is affixed (e.g., via the adhesive layer 134, without limitation) to the skin of the user via one or more couplings, each including one or more coupling elements. In various embodiments, the one or more couplings are chosen from snap connections and magnetic connections. The one or more couplings include a combined coupling force sufficient to maintain the coupling while the deployment inserter 700 is in the standard, pre-actuated, condition and during the actuation process, and is less than the force required to remove the cradle 130 from the skin of the user-body (e.g., the combined coupling force is less than the adhesive strength of the adhesive layer 134 attached to the skin of the user-body, without limitation).

In some of these various embodiments, the snap connections are formed via the bracket 138 of the cradle 130 including bracket openings 140 on each side of the bracket 138 and coupling protrusions 750 extending outward from the driver plate brackets 748, which are configured to align and snap into place within the bracket openings 140. Alternatively, or in combination, the bracket openings 140 are formed on the driver plate brackets 748 and the coupling protrusions 750 extend from the bracket 138.

In some of these various embodiments, the bracket 138 includes a ferrous material (e.g., an electromagnetic material) and the driver plate 734 includes one or more magnets 758 arranged to magnetically attract the ferrous material. Alternatively, or in combination, the driver plate 734 includes the ferrous material and the bracket 138 includes the one or more magnets 758.

In various embodiments, the driver plate 734 includes one or more protruding portions 756 extending toward the cradle 130 and configured to contact the cradle 130. The one or more protruding portions 756 may stabilize the contact between the driver plate 734 and the cradle 130. In some of these various embodiments, the thickness of the one or more protruding portions 756 is substantially the same as a height of the lip 142 and/or a height difference between the base structure 132 and the sensor housing 144. In the embodiments illustrated, the one or more magnets 758 are positioned in the one or more protruding portions 756 and the one or more protruding portions 756 are positioned to align with the bracket 138 of the cradle 130.

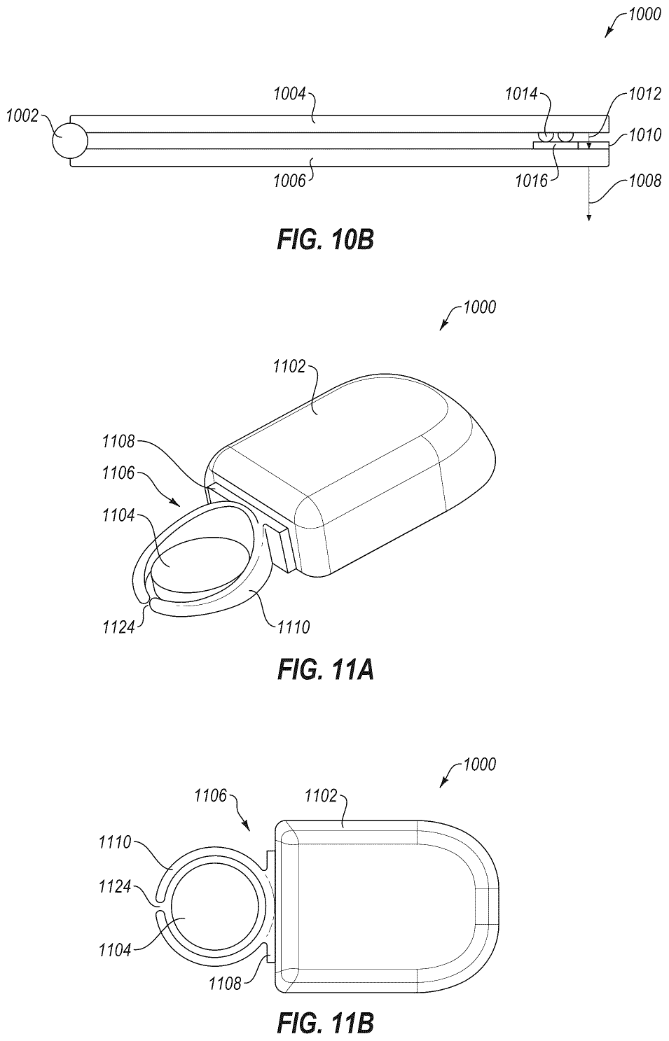

FIG. 10A is a schematic illustration of an exemplary embodiment of a fluid path integrity switch 1000 in an open condition. FIG. 10B is a schematic illustration of an exemplary embodiment of the fluid path integrity switch 1000 of FIG. 10A in a closed condition. As described above, the cradle 130 may include a cannula hub 148 that includes the cannula 150. Upon connecting the automated medicament delivery system 102 to the cradle 130 a fluid connection is made between the cannula 150 and the reservoir 112 at the cannula hub 148. The fluid path integrity switch 1000 is configured to confirm proper formation of the fluid path to prevent leakage of the medicament and to ensure proper delivery of the medicament to the user-body.

In various embodiments, the fluid path integrity switch 1000 is positioned within the cannula hub 148. While the fluid path integrity switch 1000 is illustrated with a first arm 1004, a second arm 1006, and a hinge 1002, other configurations are also contemplated, such as the first arm 1004 being a portion of the automated medicament delivery system 102 and the second arm 1006 being a portion of the cradle 130, the first arm 1004 and the second arm 1006 being respective features of the automated medicament delivery system 102 and the cradle 130 that are configured to mate together, and the fluid path integrity switch 1000 is configured to confirm the mating thereof.

The fluid path integrity switch 1000 includes ferrous material 1016 positioned on one of the first arm 1004 and the second arm 1006. The fluid path integrity switch 1000 also includes one or more magnets 1014 positioned on the other of the first arm 1004 and the second arm 1006 corresponding to the ferrous material 1016. The ferrous material 1016 may be any material that magnetically connects with the one or more magnets 1014 (e.g., a ferromagnetic material, without limitation). The one or more magnets 1014 and the ferrous material 1016 may be configured to guide the formation of the fluid coupling between the cannula 150 and the reservoir 112 at the cannula hub 148.

In various embodiments, the first arm 1004 (e.g., a fluid connector of the automated medicament delivery system 102) includes a needle 1012 fluidly connected to the reservoir 112 (FIG. 5C and FIG. 6C) and the second arm 1006 (e.g., a portion of the cannula hub 148) includes a mating portion 1010 in fluid communication with the cannula 1008 and configured to receive the needle 1012. In some of these various embodiments, the mating portion 1010 includes a mating septum and the needle 1012 is configured to be inserted to at least a predetermined amount therein. The connection between the one or more magnets 1014 and the ferrous material 1016 is configured to confirm that the needle 1012 is inserted into the mating portion 1010 to at least the predetermined amount. The fluid path integrity switch 1000 may be configured to send a signal to the controller of the automated medicament delivery system 102 to confirm that a proper fluid connection is made between the automated medicament delivery system 102 and the cradle 130. Any type of connection detection may be used to send this signal including a Hall effect, an optical sensor, and the like.

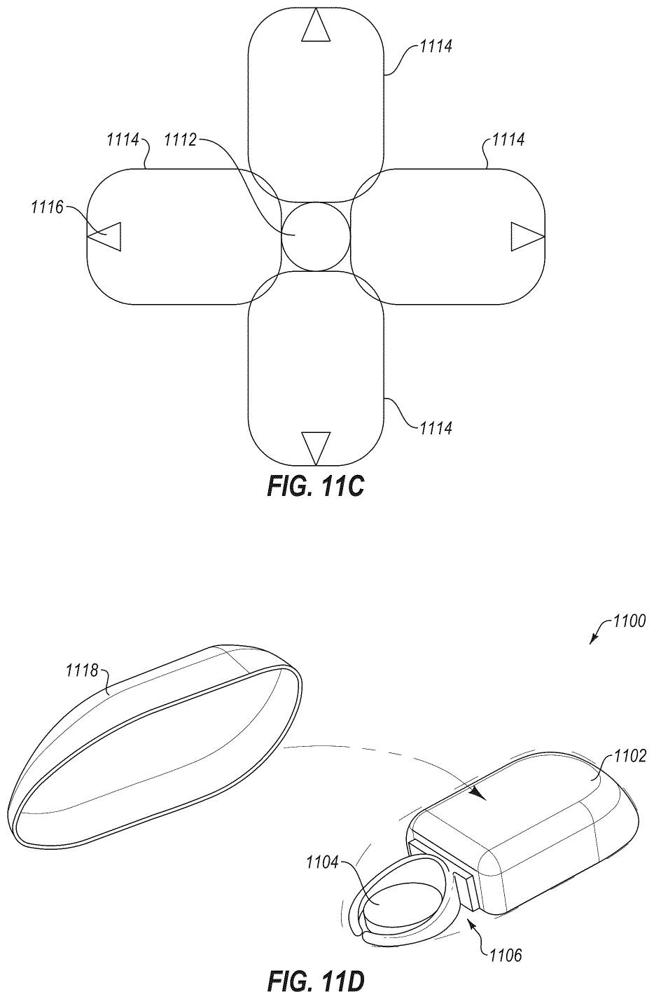

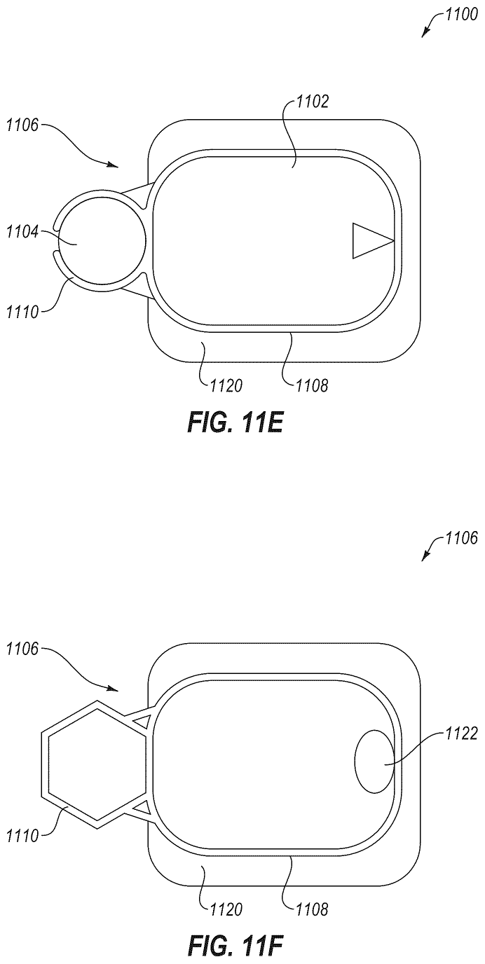

FIGS. 11A-11F are perspective views of various embodiments of a delivery and analyte assembly 1100 of the present disclosure. FIG. 11B is a top view of the delivery and analyte assembly 1100 of FIG. 11A. Referring to FIG. 11A and FIG. 11B, in various embodiments, the delivery system mount 1108 of the cradle 1106 is a connector configured to affix the cradle 1106 to the automated medicament delivery system 1102, and the sensor housing 1110 includes an annular shape configured to surround the analyte sensor 1104. In some of these embodiments, the sensor housing 1110 includes a gap 1124 formed therein. The gap 1124 may allow the sensor housing 1110 to flex around the analyte sensor 1104, which may allow the cradle 1106 to be used with analyte sensors 122 of varied sizes.

FIG. 11C is a schematic illustration of positioning for the delivery and analyte assembly 1100 of FIG. 11A. The timing that each of the analyte sensor 1104 and the automated medicament delivery system 1102 remain attached to the body may be different, and, in particular, the analyte sensor 1104 may remain on the body longer than the automated medicament delivery system 1102. Subsequently, an automated medicament delivery system 102 may be affixed to the body of the user in a different location, clocked relative to the previous position. FIG. 11C illustrates an analyte sensor position 1112 with various automated medicament delivery system positions 1114 surrounding the analyte sensor position 1112. The cannula position 1116 in each configuration may be opposite the analyte sensor position 1112.

FIG. 11D illustrates is a perspective view of the delivery and analyte assembly 1100 of FIG. 11A including a cover 1118. Referring to FIG. 11D, in various embodiments, the delivery and analyte assembly 1100 includes a cover 1118 configured to surround each of the automated medicament delivery system 1102, analyte sensor 1104, and cradle 1106 while each of the automated medicament delivery system 1102, analyte sensor 1104, and cradle 1106 are secured to the user-body. The cover 1118 may protect the automated medicament delivery system 1102 and the analyte sensor 1104 and may improve an overall appearance of the delivery and analyte assembly 1100.

FIG. 11E is a top view of a configuration of the delivery and analyte assembly 1100 of FIG. 11A. Referring to FIG. 11E, in various embodiments, the delivery system mount 1108 is configured to surround a perimeter of the automated medicament delivery system 1102. The delivery system mount 1108 may be secured to the automated medicament delivery system 1102 via a snap or interference fit.

FIG. 11F is a top view of a configuration of the cradle 1106 of FIG. 11A. Referring to FIG. 11F, in various embodiments, the sensor housing 1110 may include a different shape that corresponds to the shape of the analyte sensor 1104 or that is configured to secure the analyte sensor 1104 therein. The adhesive layer 1120 may include an adhesive layer opening 1122 formed therein that is positioned to align with the cannula of the automated medicament delivery system 1102.

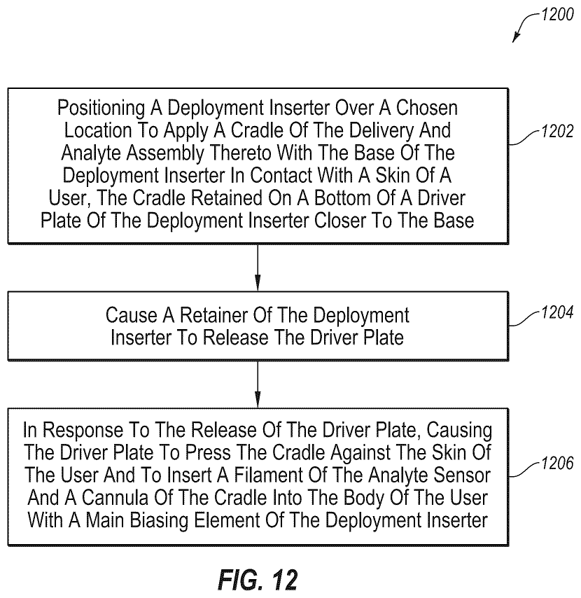

FIG. 12 is a flowchart of a method 1200 for applying a delivery and analyte assembly to user-body. The method 1200 includes positioning a deployment inserter over a chosen location to apply a cradle of the delivery and analyte assembly thereto with the base of the deployment inserter in contact with a skin of a user-body, the cradle retained on a bottom of a driver plate of the deployment inserter closer to the base at act 1202. The deployment inserter may be any of the deployment inserters disclosed herein, and the cradle may be any of the cradles disclosed herein. In various embodiments, an analyte sensor is secured to the cradle prior to the cradle being secured within the deployment inserter.

The method also includes causing a retainer of the deployment inserter to release the driver plate at act 1204. In various embodiments, the retainer is caused to release the driver plate by exerting an external force on the retainer. In some of these embodiments, the external force is applied by actuating an actuator that deflects a retainer body of the retainer and results in the release of the driver plate. For example, as described above, the driver plate may include one or more retention clips that are released due to the deflection of the retainer body.

In response to the release of the driver plate, causing the driver plate to press the cradle against the skin of the user-body and to insert a filament of the analyte sensor and a cannula of the cradle into the body of the user with the main biasing element of the deployment inserter at act 1206. The main biasing element may be in a compressed/loaded condition, which uncompressed/unloads once the driver plate is released by the retainer, resulting in the cradle being pressed against the skin of the user-body securing the cradle to the skin of the user-body by the adhesive layer, a filament sharp inserting the filament into the user-body, and a cannula sharp inserting the cannula into the user-body.

In various embodiments, the method 1200 further includes withdrawing the filament sharp and the cannula sharp from the user-body in response to the driver plate being substantially deployed, such as about fully deployed. In some of these embodiments, a filament lock beam and a cannula lock beam are released, resulting in a release of the filament sharp and the cannula sharp, respectively. A compressed filament biasing element and a compressed cannula biasing element may then press the filament sharp and the cannula sharp respectively in a direction opposite the movement of the driver plate, away from the skin of the user-body, causing the filament sharp and the cannula sharp to withdraw from the user-body.

In various embodiments, the method 1200 further includes removing a seal from the base prior to act 1202.

In various embodiments, the method 1200 further includes separating the deployment inserter from the cradle after the cradle is secured to the skin of the user and, in particular, separating the driver plate from the cradle. Separating the deployment inserter from the cradle may also be performed after the filament sharp and the cannula sharp are withdrawn from the user-body. After separating the deployment inserter from the cradle, the method 1200 further includes attaching the automated medicament delivery system to the cradle. Attaching the automated medicament delivery system to the cradle includes forming a fluid connection between the cannula and the reservoir.

The method 1200 may include any of the configurations and actions of the various embodiments of the deployment inserters and the various components thereof disclosed herein.

Non-limiting illustrative embodiments of this disclosure may include:

Embodiment 1: An assembly, comprising: an analyte sensor configured to obtain data about analyte in a user-body; an automated medicament delivery system configured to administer medicament to the user-body, the automated medicament delivery system including a reservoir configured to hold the medicament therein; and a cradle including: a sensor housing configured to at least partially encapsulate the analyte sensor while the cradle is secured to the user-body; and a delivery system mount configured to secure the automated medicament delivery system to the cradle.

Embodiment 2: The assembly according to Embodiment 1, further comprising a cannula and a cannula hub, the cannula hub configured to at least partially fluidly couple the cannula and the reservoir of the automated medicament delivery system.

Embodiment 3: The assembly according to any of Embodiments 1 and 2, wherein the automated medicament delivery system comprises a base structure including a hub receiver defining a cavity with the cannula hub received therein, a conduit fluidly coupled to the reservoir, and a mating needle extending from the conduit into the cavity and fluidly coupling with the cannula forming a fluid connection between the reservoir and the cannula.

Embodiment 4: The assembly according to any of Embodiments 1 through 3, wherein the cradle includes a seal positioned within the cannula hub and the mating needle extends through the seal to fluidly couple with the cannula.

Embodiment 5: The assembly according to any of Embodiments 1 through 4, further comprising a fluid path integrity switch configured to confirm a proper formation of a fluid coupling between the cannula and the reservoir to prevent leakage of the medicament and to ensure proper delivery of the medicament to the user-body.

Embodiment 6: The assembly according to any of Embodiments 1 through 5, wherein the fluid path integrity switch includes one or more magnets and a ferrous material configured to guide a formation of the fluid coupling between the cannula and the reservoir at the cannula hub.

Embodiment 7: The assembly according to any of Embodiments 1 through 6, wherein the sensor housing is at least partially inboard of the delivery system mount.

Embodiment 8: The assembly according to any of Embodiments 1 through 7, wherein the sensor housing is at least partially outboard of the delivery system mount.

Embodiment 9: The assembly according to any of Embodiments 1 through 8, wherein the cradle includes a base structure and an adhesive layer configured to adhere the base structure to the user-body, and wherein the sensor housing and the delivery system mount are positioned on the base structure.

Embodiment 10: The assembly according to any of Embodiments 1 through 9, wherein the sensor housing includes a sensor hole configured to receive a filament therein.

Embodiment 11: The assembly according to any of Embodiments 1 through 10, wherein the sensor housing includes a sensor opening configured to receive a filament sharp of a deployment inserter, the filament sharp configured to position a tip of a filament subcutaneously in the user-body.

Embodiment 12: A deployment inserter for a cradle of a delivery and analyte assembly, comprising: a body structure including a base defining an opening to an interior of the body structure, the base configured to contact a user-body during deployment of the cradle; a driver plate configured to retain the cradle on a bottom thereof, the cradle positioned closer to the base than the driver plate while in a pre-actuated condition; a retainer secured to the body structure and configured to hold the driver plate in position relative to and within the body structure while in the pre-actuated condition; a main biasing element in a biased condition while in the pre-actuated condition and configured to exert a force on the driver plate while in the pre-actuated condition; a filament sharp configured to insert a filament into the user-body; and a cannula sharp configured to insert a cannula into the user-body, the retainer configured to, responsive to an external force, release the driver plate and the force exerted by the main biasing element upon release of the driver plate causes the driver plate to move with the cradle in a first direction towards the base resulting in an adhesive layer of the cradle being adhered to a skin of the user-body, the filament sharp inserting the filament into the user-body, and the cannula sharp inserting the cannula into the user-body.

Embodiment 13: The deployment inserter according to Embodiment 12, further comprising an actuator configured to apply the external force to the retainer and initiate the retainer to release the driver plate.

Embodiment 14: The deployment inserter according to any of Embodiments 12 and 13, wherein the driver plate includes one or more retention clips that overlap with and secure the driver plate to the retainer and the retainer is configured to, upon actuation of the actuator, release the one or more retention clips.

Embodiment 15: The deployment inserter according to any of Embodiments 12 through 14, wherein the retainer includes a retainer base defining a retainer opening and the actuator includes an actuator wedge extending into the retainer opening, and wherein the actuator is configured to move inward causing the actuator wedge to move further into the retainer opening applying the external force thereto resulting in each side of the retainer base being separated and sides of a retainer body moving further apart initiating the release of the one or more retention clips.

Embodiment 16: The deployment inserter according to any of Embodiments 12 through 15, wherein the driver plate includes one or more coupling elements for defining one or more couplings between the driver plate and the cradle chosen from a snap connection and a magnetic connection.

Embodiment 17: The deployment inserter according to any of Embodiments 12 through 16, wherein the one or more coupling elements are chosen from a coupling protrusion configured to snap into a bracket opening formed in a bracket of the cradle and one or more magnets configured to magnetically couple with the bracket.

Embodiment 18: The deployment inserter according to any of Embodiments 12 through 17, wherein the one or more couplings between the driver plate and the cradle are configured with a combined coupling force sufficient to maintain a coupling between the driver plate and the cradle while in the pre-actuated condition and during an actuation process, and the combined coupling force is less than a force required to remove the cradle from a skin of the user-body.

Embodiment 19: The deployment inserter according to any of Embodiments 12 through 18, wherein the driver plate includes a plate body, a filament sharp guide extending from the plate body in a second direction opposite the first direction, a cannula sharp guide extending from the plate body in the second direction, a filament sharp lock configured to retain a filament sharp head of the filament sharp within the filament sharp guide until the filament is inserted into the user-body, and a cannula sharp lock configured to retain a cannula sharp head within the cannula sharp guide until the cannula is inserted into the user-body.

Embodiment 20: The deployment inserter according to any of Embodiments 12 through 19, further comprising: a filament biasing element positioned within the filament sharp guide and configured to withdraw the filament sharp from the user-body upon release of the filament sharp lock; and a cannula biasing element positioned within the cannula sharp guide and configured to withdraw the cannula sharp from the user-body upon release of the cannula sharp lock.

Embodiment 21: The deployment inserter according to any of Embodiments 12 through 20, wherein the body structure includes a first actuation guide and a second actuation guide, and each of the filament sharp lock and the cannula sharp lock includes a lock body and a lock arm, the lock arm of the filament sharp lock configured to retain the filament sharp head and the lock arm of the cannula sharp lock configured to retain the cannula sharp head, wherein the lock body of the filament sharp lock is positioned between the second actuation guide and the filament sharp guide and the lock body of the cannula sharp lock is positioned between the first actuation guide and the cannula sharp guide while in the pre-actuated condition and until the cradle is substantially deployed, and wherein the second actuation guide releases the lock body of the filament sharp lock and the first actuation guide releases the lock body of the cannula sharp lock once the cradle is substantially deployed.

Embodiment 22: The deployment inserter according to any of Embodiments 12 through 21, wherein upon release of the lock body of the filament sharp lock the filament biasing element pushes the filament sharp head in the second direction, pushing the lock arm of the filament sharp lock out from over a sharp guide end of the filament sharp guide and withdrawing the filament sharp from the user-body, and upon release of the lock body of the cannula sharp lock the cannula biasing element pushes the cannula sharp head in the second direction, pushing the lock arm of the cannula sharp lock out from over a sharp guide end of the cannula sharp guide and withdrawing the cannula sharp from the user-body.

Embodiment 23: The deployment inserter according to any of Embodiments 12 through 22, wherein the driver plate, filament sharp, and cannula sharp are configured to cause each of the filament and the cannula to be inserted into the user-body at about the same time.

Embodiment 24: A system, comprising: an automated medicament delivery system configured to administer medicament to a user-body, the automated medicament delivery system including a reservoir configured to hold the medicament therein; a cradle assembly including: an analyte sensor configured to obtain data about analyte in a user-body, the analyte sensor including a filament; and a cradle including a sensor housing configured to secure the analyte sensor to the cradle and position the filament relative to the user-body while the cradle is secured to the user-body, a delivery system mount configured to secure the automated medicament delivery system to the cradle, and an adhesive layer configured to adhere the cradle to the user-body; and a deployment inserter including: a body structure including a base defining an opening to an interior of the body structure, the base configured to contact the user-body during deployment of the cradle assembly; a driver plate with the cradle retained on a bottom thereof, the cradle positioned closer to the base than the driver plate while in a pre-actuated condition of the deployment inserter; a retainer secured to the body structure and configured to hold the driver plate in position relative to and within the body structure while in the pre-actuated condition; a main biasing element in a biased condition while in the pre-actuated condition and configured to exert a force on the driver plate while in the pre-actuated condition; a filament sharp configured to insert the filament into the user-body; a cannula sharp configured to insert the cannula into the user-body; and the retainer configured to, responsive to an external force, release the driver plate and the force exerted by the main biasing element upon release of the driver plate causes the driver plate to move with the cradle in a first direction towards the base resulting in the adhesive layer of the cradle being adhered to a skin of the user-body, the filament sharp inserting the filament into the user-body, and the cannula sharp inserting the cannula into the user-body.

Embodiment 25: A method for applying an assembly to a user-body, comprising: positioning a deployment inserter over a chosen location to apply a cradle of the assembly thereto with a base of the deployment inserter in contact with a skin of the user-body, the cradle retained on a bottom of a driver plate of the deployment inserter closer to the base; causing a retainer of the deployment inserter to release the driver plate; and in response to the release of the driver plate, causing the driver plate to press the cradle against the skin of the user-body and to insert a filament and a cannula into the user-body with a main biasing element of the deployment inserter.

Embodiment 26: The method according to Embodiment 25, wherein causing the retainer to release the driver plate includes applying an external force to a retainer body of the retainer to deflect the retainer body and release the driver plate.

Embodiment 27: The method according to any of Embodiments 25 and 26, wherein applying the external force includes actuating an actuator that applies the external force to the retainer body.

Embodiment 28: The method according to any of Embodiments 25 through 27, further comprising securing an analyte sensor to the cradle prior to the cradle being positioned within the deployment inserter and retained by the driver plate.

Embodiment 29: The method according to any of Embodiments 25 through 28, further comprising withdrawing a filament sharp and a cannula sharp from the user-body in response to the driver plate being substantially deployed and after the filament sharp inserted the filament into the user-body and the cannula sharp inserted the cannula into the user-body.

Embodiment 30: The method according to any of Embodiments 25 through 29, wherein withdrawing the filament sharp and the cannula sharp includes releasing a filament lock beam and a cannula lock beam, resulting in a release of the filament sharp and the cannula sharp respectively once the driver plate is substantially deployed.

Embodiment 31: The method according to any of Embodiments 25 through 30, wherein a compressed filament biasing element and a compressed cannula biasing element press the filament sharp and the cannula sharp respectively in a direction opposite movement of the driver plate, away from the user-body, causing the filament sharp and the cannula sharp to withdraw from the user-body.

Embodiment 32: The method according to any of Embodiments 25 through 31, further comprising removing a seal from the base prior to positioning the deployment inserter over a chosen location.

Embodiment 33: The method according to any of Embodiments 25 through 32, further comprising separating the deployment inserter from the cradle after the cradle is secured to the skin of the user-body.

Embodiment 34: The method according to any of Embodiments 25 through 33, further comprising attaching an automated medicament delivery system of the assembly to the cradle including forming a fluid connection between the cannula and a reservoir of the automated medicament delivery system.

Embodiment 35: The method according to any of Embodiments 25 through 34, wherein each of the filament and the cannula to be inserted into the user-body at about the same time.

Embodiment 36: An apparatus, comprising: a base structure including a first side configured to sit on a user-body and a second side configured to receive a device to administer medicament to a user-body; and a hub formed in the base structure, the hub configured to fluidly couple the first side of the base structure to the second side of the base structure.

Embodiment 37: The apparatus according to Embodiment 36, further comprising a seal in a position chosen from among an outer surface of the hub and an inner surface of the hub, the seal configured to form a fluid tight seal between the hub and an automated medicament delivery system.

Embodiment 38: The apparatus according to any of Embodiments 36 and 37, further comprising a cannula fluidly coupled with the hub and extending from an interior of the hub to beyond the first side.

Embodiment 39: The apparatus according to any of Embodiments 36 through 38, wherein the hub includes: a cylindrical body at the second side and extending away from the first side; and a through hole defined by the has structure extending from the first side to the second side and through the cylindrical body.

Embodiment 40: The apparatus according to any of Embodiments 36 through 39, further comprising a valve in the hub configured to selectively control a fluidic connection between the apparatus and an automated medicament delivery system.

The embodiments described above and illustrated in the accompanying drawings do not limit the scope of the disclosure, which is encompassed by the scope of the appended claims and their legal equivalents. Any equivalent embodiments are within the scope of this disclosure. Indeed, various modifications, in addition to those shown and described herein, such as alternate useful combinations of the elements described, will become apparent to those skilled in the art from the description. Such modifications and embodiments also fall within the scope of the appended claims and equivalents.

What is claimed is:

1. An assembly, comprising:
an analyte sensor configured to obtain data about analyte in a user-body;
an automated medicament delivery system configured to administer medicament to the user-body, the automated medicament delivery system including a reservoir configured to hold the medicament therein; and
a cradle including:
a sensor housing configured to at least partially encapsulate the analyte sensor while the cradle is secured to the user-body; and
a delivery system mount configured to secure the automated medicament delivery system to the cradle.

2. The assembly of claim 1, wherein the cradle comprises a cannula and a cannula hub, the cannula hub configured to at least partially fluidly couple the cannula and the reservoir of the automated medicament delivery system.

3. The assembly of claim 2, wherein the automated medicament delivery system comprises a base structure including a hub receiver defining a cavity with the cannula hub received therein, a conduit fluidly coupled to the reservoir, and a mating needle extending from the conduit into the cavity and fluidly coupling with the cannula forming a fluid connection between the reservoir and the cannula.

4. The assembly of claim 3, wherein the cradle includes a seal positioned within the cannula hub and the mating needle extends through the seal to fluidly couple with the cannula.

5. The assembly of claim 3, further comprising a fluid path integrity switch configured to confirm a proper formation of a fluid coupling between the cannula and the reservoir to prevent leakage of the medicament and to ensure proper delivery of the medicament to the user-body.

6. The assembly of claim 5, wherein the fluid path integrity switch includes one or more magnets and a ferrous material configured to guide a formation of the fluid coupling between the cannula and the reservoir at the cannula hub.

7. The assembly of claim 1, wherein the cradle includes a base structure and an adhesive layer configured to adhere the base structure to the user-body, and wherein the sensor housing and the delivery system mount are positioned on the base structure.

8. The assembly of claim 1, wherein the sensor housing includes a sensor hole configured to receive a filament therein.

9. The assembly of claim 1, wherein the sensor housing includes a sensor opening configured to receive a filament sharp of a deployment inserter, the filament sharp configured to position a tip of a filament subcutaneously in the user-body.

10. A system, comprising:
an assembly comprising:
an analyte sensor configured to obtain data about analyte in a user-body;
an automated medicament delivery system configured to administer medicament to the user-body, the automated medicament delivery system including a reservoir configured to hold the medicament therein; and
a cradle including:
a sensor housing configured to at least partially encapsulate the analyte sensor while the cradle is secured to the user-body; and
a delivery system mount configured to secure the automated medicament delivery system to the cradle; and
a deployment inserter comprising:
a body structure including a base defining an opening to an interior of the body structure, the base configured to contact a user-body during deployment of the cradle;
a driver plate configured to retain the cradle on a bottom thereof, the cradle positioned closer to the base than the driver plate while in a pre-actuated condition;
a retainer secured to the body structure and configured to hold the driver plate in position relative to and within the body structure while in the pre-actuated condition;
a main biasing element in a biased condition while in the pre-actuated condition and configured to exert a force on the driver plate while in the pre-actuated condition;
a filament sharp configured to insert the filament into the user-body; and
a cannula sharp configured to insert a cannula into the user-body,
the retainer configured to, responsive to an external force, release the driver plate and the force exerted by the main biasing element upon release of the driver plate causes the driver plate to move with the cradle in a first direction toward the base resulting in an adhesive layer of the cradle being adhered to a skin of the user-body, the filament sharp inserting the filament into the user-body, and the cannula sharp inserting the cannula into the user-body.

11. The system of claim 10, further comprising an actuator configured to apply the external force to the retainer and initiate the retainer to release the driver plate.

12. The system of claim 10, wherein the driver plate includes one or more retention clips that overlap with and secure the driver plate to the retainer and the retainer is configured to, upon actuation of the actuator, release the one or more retention clips.

13. The system of claim 10, wherein the retainer includes a retainer base defining a retainer opening and the actuator includes an actuator wedge extending into the retainer opening, and wherein the actuator is configured to move inward causing the actuator wedge to move further into the retainer opening applying the external force thereto resulting in each side of the retainer base being separated and sides of a retainer body moving further apart initiating the release of the one or more retention clips.

14. The deployment inserter of claim 10, wherein the driver plate, filament sharp, and cannula sharp are configured to cause each of the filament and the cannula to be inserted into the user-body at about the same time.

15. A method for applying the assembly of claim 10 to a user-body, comprising:
positioning the deployment inserter over a chosen location to apply the cradle of the assembly thereto with the base of the deployment inserter in contact with a skin of the user-body, the cradle retained on a bottom of a driver plate of the deployment inserter closer to the base;
causing the retainer of the deployment inserter to release the driver plate; and
in response to the release of the driver plate, causing the driver plate to press the cradle against the skin of the user-body and to insert the filament and the cannula into the user-body with the main biasing element of the deployment inserter.

16. The method of claim 15, further comprising securing the analyte sensor to the cradle prior to the cradle being positioned within the deployment inserter and retained by the driver plate.

17. The method of claim 15, further comprising withdrawing a filament sharp and a cannula sharp from the user-body in response to the driver plate being substantially deployed and after the filament sharp inserted the filament into the user-body and the cannula sharp inserted the cannula into the user-body.

18. The method of claim 15, further comprising removing a seal from the base prior to positioning the deployment inserter over a chosen location.

19. The method of claim 15, further comprising separating the deployment inserter from the cradle after the cradle is secured to the skin of the user-body.

20. The method of claim 19, wherein the cradle comprises the cannula, the method further comprising attaching the automated medicament delivery system to the cradle including forming a fluid connection between the cannula and the reservoir of the automated medicament delivery system.
